SEQUENCE LISTING (48 sequences)

SEQ ID NO: 1 - DNA, 1092 bp, Legionella pneumophila
  source 1..1092: mol_type = other DNA
ctaacaatcatttttttgcttaatatccgaccatttaatgggttcacctatagataccttgttttgtaattcccagccatttaccagatcccagtattgggtcgggactcccttacaaggaaaggcaaaggttgtattatcaaggctaattagagagccaggttgaagtttttgctttgcaaccaaacaagttctttgagagctgccaagtactccttttataggagcttctagatctcgcatcggttgacctaaagcttgccaacaattgtaaattgtttcaagagtcttggctaattgctcaatccccatagtatgtgatatgtcctgatcaatatcttctggatagagatgaactcctttttccaggattgaagctcccaatgcaatagacatatacaacatttcaattccattataatggtcagataaaccaactggtaatgaaaaacattggtttaaagttcgtaacattcttaggttatgtgcttctggcaaagcaggatgtccatcaggactatgttcaataataattttttcacatccggcactttttgctgtttcaactgctttgcatatatcagttaaagaagatcttccagtgtcaataatgagcggtaatcctgtttttgccagatgacgaattaaaggaacatggacaatgttcgatgaagaggttttgagtgcgcaggcaccctgctttatcgcaaaatcaatagtgtccaattcataaatcgaaactatgaagggtaaattggcattctttaatagtgaaaatagcatactataatgttctaaaggcagtacttttctctcaattaatgagcggtagttttccttgcggacctcaccggatttactggtataggtttcttcataatcaacaggtaggcaaatgtttgggttatttaaaatttctgtttttagtataagaggttggtatggtactttatccctgatggttataattttgtgtagcaattgttcagctatgccaagatcctgattaaaaaaagtacctatttctgctaaaaaaataggaggatgattttcttcaacagagaatggaccaattgtaaaaggctgactcat

SEQ ID NO: 2 - DNA, 19 bp, unidentified
  misc_feature 1..19: note = Synthetic oligonucleotide
  source 1..19: mol_type = other DNA
agctcccaatgcaatagac

SEQ ID NO: 3 - DNA, 19 bp, unidentified
  misc_feature 1..19: note = Synthetic oligonucleotide
  source 1..19: mol_type = other DNA
gcaaagcagttgaaacagc

SEQ ID NO: 4 - DNA, 25 bp, unidentified
  misc_feature 1..25: note = Synthetic oligonucleotide
  source 1..25: mol_type = other DNA
ccaactggtaatgaaaaacattggt

SEQ ID NO: 5 - DNA, 405 bp, Legionella pneumophila
  source 1..405: mol_type = other DNA
ggggcgacctggcttcgacgtgggttgcaaaaccggaagtgcatgccgagaaggagatctctcgtaaataagactcaattaaatataaatgcaaacgatgaaaactttgctggtggggaagctatcgctgcctaataagcactttagttaaaccatcactgtgtactggccaataaacccagtatcccgttcgaccgagcccgcttatcggtatcgaatcaacggtcataagagataagctagcgtcctaatctatcccgggttatggcgcgaaactcagggaatcgctgtgtatcatcctgcccgtcggaggagccacagttaaattcaaaagacaaggctatgcatgtagagctaaaggcagaggacttgcggacgcgggttcgattcccgccgcctccacca

SEQ ID NO: 6 - DNA, 471 bp, Legionella pneumophila
  source 1..471: mol_type = other DNA
atgactaccaaaaaacaaccagattccaccatagccttgaatagaaaagctggttttgattactttattgaagatcaatacgaagcaggcctggttttggaaggctgggaagtaaaaagtctgcgtgctggaaaaatcaatttgtcggattcacacgtgataatcaaatacggtgaggcattcctattgggcgcccaaatacagcctcttcccaccgcatccactcattttattcctgatccgatcaggacgcgcaagctattgatgaataaaaaagaattaaaccatctcataggaagtgttgaaaggcaaggctataccatagtccctctttctttgtattggaaaaaaaataaaattaaaataaaaattgctctggctaaaggaaaaaaagagcatgacaaaagagacacgatcaaagacagagaatggcaaagggatcgttcaagaataatgaaaaagaacacttga

SEQ ID NO: 7 - DNA, 21 bp, unidentified
  misc_feature 1..21: note = Synthetic oligonucleotide
  source 1..21: mol_type = other DNA
cacgtgataatmaaatacggt

SEQ ID NO: 8 - DNA, 19 bp, unidentified
  misc_feature 1..19: note = Synthetic oligonucleotide
  source 1..19: mol_type = other DNA
ttcatcaatagcttgcgyg

SEQ ID NO: 9 - DNA, 26 bp, unidentified
  misc_feature 1..26: note = Synthetic oligonucleotide
  source 1..26: mol_type = other DNA
cygcatccactcattttattcctgat

SEQ ID NO: 10 - DNA, 14 bp, unidentified
  misc_feature 1..14: note = Synthetic oligonucleotide
  source 1..14: mol_type = other DNA
ggcgacctggcttc

SEQ ID NO: 11 - DNA, 21 bp, unidentified
  misc_feature 1..21: note = Synthetic oligonucleotide
  source 1..21: mol_type = other DNA
tatgaccgttgattcgatacc

SEQ ID NO: 12 - DNA, 17 bp, unidentified
  misc_feature 1..17: note = Synthetic oligonucleotide
  source 1..17: mol_type = other DNA
acgtgggttgcraaacc

SEQ ID NO: 13 - DNA, 19 bp, unidentified
  misc_feature 1..19: note = Synthetic oligonucleotide
  source 1..19: mol_type = other DNA
ttcatcaacagcttgcgtg

SEQ ID NO: 14 - DNA, 18 bp, unidentified
  misc_feature 1..18: note = Synthetic oligonucleotide
  source 1..18: mol_type = other DNA
gatcaatacgaagcaggc

SEQ ID NO: 15 - DNA, 20 bp, unidentified
  misc_feature 1..20: note = Synthetic oligonucleotide
  source 1..20: mol_type = other DNA
gccattctctgtctttgatc

SEQ ID NO: 16 - DNA, 22 bp, unidentified
  misc_feature 1..22: note = Synthetic oligonucleotide
  source 1..22: mol_type = other DNA
ccaagtagggtttctaacaatc

SEQ ID NO: 17 - DNA, 21 bp, unidentified
  misc_feature 1..21: note = Synthetic oligonucleotide
  source 1..21: mol_type = other DNA
gagtcagccttttacaattgg

SEQ ID NO: 18 - DNA, 19 bp, unidentified
  misc_feature 1..19: note = Synthetic oligonucleotide
  source 1..19: mol_type = other DNA
atgccagtcagcataagga

SEQ ID NO: 19 - DNA, 21 bp, unidentified
  misc_feature 1..21: note = Synthetic oligonucleotide
  source 1..21: mol_type = other DNA
cagacctctggtaggatgtac

SEQ ID NO: 20 - DNA, 20 bp, unidentified
  misc_feature 1..20: note = Synthetic oligonucleotide
  source 1..20: mol_type = other DNA
tcggcactaccgacacgaac

SEQ ID NO: 21 - DNA, 1091 bp, Legionella pneumophila
  source 1..1091: mol_type = other DNA
taacaatcatttttttgcttaatatccgaccatttaatgggttcacctatagataccttgttttgtaattcccagccatttaccagatcccagtattgggtaggaactcccttacaaggaaaggcaaaggttgtattatcaaggctaattagagagccgggttgaagtttttgctttgcaaccaaacaagttctttgagagctgccaagtactccttttataggggtttctagatctcgcattggttgacctaaagcttgccaacaattgtaaattgtttcaagagtcttggctaattgatcaatccccatagtatgtgatatgtcctgatcgatatcttctggatagagatgaactcctttttccaggattgaagctcccaatgcaatagacatatacaacatttcaattccattataatggtcagataaaccaactggtaatgaaaaacattggtttaaagttcgtaacattcttaggttatgtgcttctggcaaagcaggatgtccatcagggctatgttcaataataattttttcacatccggcactttttgctgtttcaactgctttgcatatatcagttaaggaagatcttccagtgtcaataatgagcggtaatcctgtttttgccagatgacgaattaaaggaatatggacaatgttcgatgaagaggttttgagtgcgcaggccccctgctttatcgcaaaatcaatagtgtccaattcataaatcgaaactatgaagggtaaattggcattctttaatagtgaaaatagcatactataatgttctaaaggcagtacttttctctcaattaatgagcggtagttttccttgcgaacctcacctgatttactggtataggtttcttcataatcaacaggtaggcaaatgtttgggttatttaaaatttctgtttttagtataagaggttggtatggtactttatccctgatggttataattttgtgtagcaattgttcagctatgccaagatcctgattaaaaaaagtacctatttctgctaaaaaaataggaggatgattttcttcaacagagaatggaccaattgtaaaaggctgactcat

SEQ ID NO: 22 - DNA, 1091 bp, Legionella pneumophila
  source 1..1091: mol_type = other DNA
taacaatcatttttttgcttaatatccgaccatttaatgggttcacctatagataccttgttttgtaattcccagccatttaccagatcccagtattgggtaggaactcccttacaaggaaaggcaaaggttgtattatcaaggctaattagagagccgggttgaagtttttgctttgcaaccaaacaagttctttgagagctgccaagtactccttttataggggtttctagatctcgcattggttgacctaaagcttgccaacaattgtaaattgtttcaagagtcttggctaattgatcaatccccatagtatgtgatatgtcctgatcgatatcttctggatagagatgaactcctttttccaggattgaagctcccaatgcaatagacatatacaacatttcaattccattataatggtcagataaaccaactggtaatgaaaaacattggtttaaagttcgtaacattcttaggttatgtgcttctggcaaagcaggatgtccatcagggctatgttcaataataattttttcacatccggcactttttgctgtttcaactgctttgcatatatcagttaaggaagatcttccagtgtcaataatgagcggtaatcctgtttttgccagatgacgaattaaaggaatatggacaatgttcgatgaagaggttttgagtgcgcaggccccctgctttatcgcaaaatcaatagtgtccaattcataaatcgaaactatgaagggtaaattggcattctttaatagtgaaaatagcatactataatgttctaaaggcagtacttttctctcaattaatgagcggtagttttccttgcgaacctcacctgatttactggtataggtttcttcataatcaacaggtaggcaaatgtttgggttatttaaaatttctgtttttagcataagaggttggtatggtactttatccctgatggttataattttgtgtagcaattgttcagctatgccaagatcctgattaaaaaaagtacctatttctgctaaaaaaataggaggatgattttcttcaacagagaatggaccaattgtaaaaggctgactcat

SEQ ID NO: 23 - DNA, 1091 bp, Legionella pneumophila
  source 1..1091: mol_type = other DNA
ctaacaatcatttttttgcttaatatccgaccatttaatgggttcacctatagataccttgttttgtaattcccacccatttaccagatcccagtattgggtcggtactcccttgcaaggaaaggcaaaggttgtattatcaaggctaattagagtaccaggatgaagttcttgttttgcaaccaaacaagttctttgagagctaccaagtactccttttatgggagcttctagatctcgcatcggttgacctaaagcttgccaacaattgtaaattgtttaagagtcttggctaattgctcaatccccatagtatgtgatatgtcctgatcaatatcttctggatagagatgaactcctttttccaggattgaagctcccaatgcaatagacatatacaacatttcaattccattataatggtcagataaaccaactggtaatgaaaaacattggtttaaagttcgtaacattcttaggttatgtgcttctggcaaagcaggatgtccatcaggactatgttcaataataattttttcacatccggcactttttgctgtttcaactgctttgcatatatcagttaaagaagatcttccagtgtcaataatgagcggtaatcctgtttttgccagatgacgaattaaaggaacatggacaatgttcgatgaagaggttttgagtgcgcaggcaccctgctttatcgcaaaatcaatagtgtccaattcataaatcgaaactatgaagggtaaattggcattctttaatagtgaaaatagcatactataatgttctaaaggcagtacttttctctcaattaatgagcggtagttttccttgcggacctcacctgatttactggtataggtttcttcataatcaacaggtaggcaaatgtttgggttatttaaaatttctgtttttagtataagaggttgatacggtactttatccctgatggttataattttgtgtagtaattgttcggctacaccaagatcctgattaaaaaaagtaccaatttctgccaaaaaaataggaggatgattttctccaatagagaatggaccaattgtaaaaggctgactcat

SEQ ID NO: 24 - DNA, 1074 bp, Legionella pneumophila
  source 1..1074: mol_type = other DNA
taacaatcatttttttgcttaatatccgaccatttaatgggttcacctatagataccttgttttgtaattcccagccatttaccagatcccagtattgggtaggaactcccttacaaggaaaggcaaaggttgtattatcaaggctaattagagagccgggttgaagtttttgctttgcaaccaaacaagttctttgagagctgccaagtactccttttataggggtttctagatctcgcattggttgacctaaagcttgccaacaattgtaaattgtttcaagagtcttggctaattgatcaatccccatagtatgtgatatgtcctgatcgatatcttctggatagagatgaactcctttttccaggattgaagctcccaatgcaatagacatatacaacatttcaattccattataatggtcagataaaccaactggtaatgaaaaacattggtttaaagttcgtaacattcttaggttatgtgcttctggcaaagcaggatgtccatcaggactatgttcaataataattttttcacatccggcactttttgctgtttcaactgctttgcatatatcagttaaggaagatcttccagtgtcaataatgagcggtaatcctgtttttgccagatgacgaattaaaggaatatggacaatgttcgatgaagaggttttgagtgcgcaggccccctgctttatcgcaaaatcaatagtgtccaattcataaatcgaaactatgaagggtaaattggcattctttaatagtgaaaatagcatactataatgttctaaaggcagtacttttctctcaattaatgagcggtagttttccttgcgaacctcacctgatttactggtataggtttcttcataatcaacaggtaggcaaatgtttgggttatttaaaatttctgtttttagtataagaggttgatacggtattttatccctgatacttataattttgtgtagcaattgttcagctatgccaagatcctgattaaaaaaagtacctatttctgctaaaaaaataggaggatgattttctccaatagagtaggggccaatt

SEQ ID NO: 25 - DNA, 1074 bp, Legionella pneumophila; identical to SEQ ID NO: 24
  source 1..1074: mol_type = other DNA

SEQ ID NO: 26 - DNA, 1074 bp, Legionella pneumophila; identical to SEQ ID NO: 24
  source 1..1074: mol_type = other DNA

SEQ ID NO: 27 - DNA, 1074 bp, Legionella pneumophila; identical to SEQ ID NO: 24
  source 1..1074: mol_type = other DNA

SEQ ID NO: 28 - DNA, 1074 bp, Legionella pneumophila; identical to SEQ ID NO: 24
  source 1..1074: mol_type = other DNA

SEQ ID NO: 29 - DNA, 1074 bp, Legionella pneumophila; identical to SEQ ID NO: 24
  source 1..1074: mol_type = other DNA

SEQ ID NO: 30 - DNA, 1074 bp, Legionella pneumophila; identical to SEQ ID NO: 24
  source 1..1074: mol_type = other DNA

SEQ ID NO: 31 - DNA, 1092 bp, Legionella pneumophila
  source 1..1092: mol_type = other DNA
ctaacaatcatttttttgcttaatatctgaccatttaatgggctcacctatagataccttgttttgtagttcccagccatttaccagatcccagtattgggtaggaactcccttacaaggaaaggcaaaggttgtattatcaaggctaattagagagccgggttgaagcttttgctttgcaaccaaacaagttctttgagagctgccaagtactccttttataggggtttctagatctcgcatcggttgacctaaagcttgccaacaattgtaaattgtttcaagagtcttggctaattgatcaatccccatagtatgtgatatgtcctgatcaatatcttctggatagagatgaacccctttttccaggattgaagctcccaatgcaatagacatatacaacatttcaattccattataatggtcagataatccaactggtaatgaaaaacattggtttaaagttcgtaacattcttaagttatgtgcttctggcaaagcaggatgtccatcaggactatgttcaataataattttttcacatccggcattttttgctgtttcaactgctttgcatatatcagttaaggaagatcttccagtgtcaataataagcggtaatcctgtttttgccagatgacgaattaaaggaatatggacaatgttcgatgaagaggttttgagtgcgcaggccccttgctttattgcaaaatcaatagtgtccaattcataaattgaaactatgaagggtaaattggcattctttaatagtgagaatagcatactgtaatgttctaaaggcagtatttttctctcaattaatgagcggtagttttccttgcgaacctcacctgatttactagtataggtttcttcataatcaacaggtaggcaaatgtttgggttatttaaaatttctgtttttagtataagaggttgatacggtactttatctctgatggttataattttgtgtagcaattgttcagctatgccaagatcctgattaaaaaaagtacctatttctgctaaaaaaataggaggatgattttctccaatagagaatggtccaattgtaaaaggctgactcat

SEQ ID NO: 32 - DNA, 1092 bp, Legionella pneumophila; identical to SEQ ID NO: 1
  source 1..1092: mol_type = other DNA

SEQ ID NO: 33 - DNA, 1092 bp, Legionella pneumophila; identical to SEQ ID NO: 1
  source 1..1092: mol_type = other DNA

SEQ ID NO: 34 - DNA, 1092 bp, Legionella pneumophila; identical to SEQ ID NO: 1
  source 1..1092: mol_type = other DNA

SEQ ID NO: 35 - DNA, 1092 bp, Legionella pneumophila; identical to SEQ ID NO: 1
  source 1..1092: mol_type = other DNA

SEQ ID NO: 36 - DNA, 1060 bp, Legionella pneumophila
  source 1..1060: mol_type = other DNA
atttttttgcttaatatccgaccatttaatgggttcacctatagataccttgttttgtaattcccagccatttaccagatcccagtattgggtcgggactcccttacaaggaaaggcaaaggttgtattatcaaggctaattagagagccaggttgaagtttttgctttgcaaccaaacaagttctttgagagctgccaagtactccttttataggagcttctagatctcgcatcggttgacctaaagcttgccaacaattgtaaattgtttcaagagtcttggctaattgctcaatccccatagtatgtgatatgtcctgatcaatatcttctggatagagatgaactcctttttccaggattgaagctcccaatgcaatagacatatacaacatttcaattccattataatggtcagataaaccaactggtaatgaaaaacattggtttaaagttcgtaacattcttaggttatgtgcttctggcaaagcaggatgtccatcaggactatgttcaataataattttttcacatccggcactttttgctgtttcaactgctttgcatatatcagttaaagaagatcttccagtgtcaataatgagcggtaatcctgtttttgccagatgacgaattaaaggaacatggacaatgttcgatgaagaggttttgagtgcgcaggcaccctgctttatcgcaaaatcaatagtgtccaattcataaatcgaaactatgaagggtaaattggcattctttaatagtgaaaatagcatactataatgttctaaaggcagtacttttctctcaattaatgagcggtagttttccttgcggacctcaccggatttactggtataggtttcttcataatcaacaggtaggcaaatgtttgggttatttaaaatttctgtttttagtataagaggttggtatggtactttatccctgatggttataattttgtgtagcaattgttcagctatgccaagatcctgattaaaaaaagtacctatttctgctaaaaaaataggaggatgattttcttcaacagagaatgga

SEQ ID NO: 37 - DNA, 1060 bp, Legionella pneumophila
  source 1..1060: mol_type = other DNA
atttttttgcttaatatctgaccatttaatgggctcacctatagataccttgttttgtagttcccagccatttaccagatcccagtattgggtaggaaatcccttacaaggaaaggcaaaggttgtattatcaaggctaattagagagccgggttgaagcttttgctttgcaaccaaacaagttctttgagagctgccaagtactccttttataggggtttctagatctcgcatcggttgacctaaagcttgccaacaattgtaaattgtttcaagagtcttggctaattgatcaatccccatagtatgtgatatgtcctgatcaatatcttctggatagagatgaacccctttttccaggattgaagctcccaatgcaatagacatatacaacatttcaattccattataatggtcagataatccaactggtaatgaaaaacattggtttaaagttcgtaacattcttaagttatgtgcttctggcaaagcaggatgtccatcaggactatgttcaataataattttttcacatccggcattttttgctgtttcaactgctttgcatatatcagttaaggaagatcttccagtgtcaataataagcggtaatcctgtttttgccagatgacgaattaaaggaatatggacaatgttcgatgaagaggttttgagtgcgcaggccccttgctttattgcaaaatcaatagtgtccaattcataaattgaaactatgaagggtaaattggcattctttaatagtgagaatagcatactgtaatgttctaaaggcagtatttttctctcaattaatgagcggtagttttccttgcgaacctcacctgatttactagtataggtttcttcataatcaacaggtaggcaaatgtttgggttatttaaaatttctgtttttagtataagaggttgatacggtactttatctctgatggttataattttgtgtagcaattgttcagctatgccaagatcctgattaaaaaaagtacctatttctgctaaaaaaataggaggatgattttctccaatagagaatggt

SEQ ID NO: 38 - DNA, 1060 bp, Legionella pneumophila; identical to SEQ ID NO: 36
  source 1..1060: mol_type = other DNA

SEQ ID NO: 39 - DNA, 1060 bp, Legionella pneumophila; identical to SEQ ID NO: 36
  source 1..1060: mol_type = other DNA

SEQ ID NO: 40 - DNA, 1060 bp, Legionella pneumophila; identical to SEQ ID NO: 36
  source 1..1060: mol_type = other DNA

SEQ ID NO: 41 - DNA, 1060 bp, Legionella pneumophila; identical to SEQ ID NO: 36
  source 1..1060: mol_type = other DNA

SEQ ID NO: 42 - DNA, 1060 bp, Legionella pneumophila; identical to SEQ ID NO: 37
  source 1..1060: mol_type = other DNA

SEQ ID NO: 43 - DNA, 1060 bp, Legionella pneumophila
  source 1..1060: mol_type = other DNA
atttttttgcttaatatctgaccatttaatgggctcacctatagataccttgttttgtagttcccagccatttaccagatcccagtattgggtaggaactcccttacaaggaaaggcaaaggttgtattatcaaggctaattagagagccgggttgaagcttttgctttgcaaccaaacaagttctttgagagctgccaagtactccttttataggggtttccagatctcgcattggttgacctaaagcttgccaacaattgtaaattgtttcaagagtcttggctaattgatcaatccccatagtatgtgatatgtcctgatcaatatcttctggatagagatgaacccctttttccaggattgaagctcccaatgcaatagacatatacaacatttcaattccattataatggtcagataatccaactggtaatgaaaaacattggtttaaagttcgtaacattcttaagttatgtgcttctggcaaagcaggatggccatcaggactatgttcaataataattttttcacatccggcattttttgctgtttcaactgctttgcatatatcagttaaggaagatcttccagtgtcaataatgagcggtaatcctgtttttgccagatgacgaattaaaggaatatggacaatgtttgatgaagaggttttgagtgcgcaggccccttgctttattgcaaaatcaatagtgtccaattcataaattgaaactatgaagggtaaattggcattctttaatagtgagaatagcatactgtaatgttctaaaggcagtatttttctctcaattaatgagcggtagttttccttgcgaacctcacctgatttactagtgtaggtttcttcataatcaacaggtaggcaaatgtttgggttatttaaaatttctgtttttagtataagaggttgatacggtactttatctctgatggttataattttgtgtagcaattgttcagctatgccaagatcctgattaaaaaaagtacctatttctgctaaaaaaataggaggatgattttctccaatagagaatggt

SEQ ID NO: 44 - DNA, 1060 bp, Legionella pneumophila; identical to SEQ ID NO: 36
  source 1..1060: mol_type = other DNA

SEQ ID NO: 45 - DNA, 1060 bp, Legionella pneumophila
  source 1..1060: mol_type = other DNA
atttttttgcttaatatccgaccatttaatgggttcacctatagataccttgttttgtaattcccagccatttaccagatcccagtattgggtaggaactcccttacaaggaaaggcaaaggttgtattatcaaggctaattagagagccgggttgaagtttttgctttgcaaccaaacaagttctttgagagctgccaagtactccttttataggggtttctagatctcgcattggttgacctaaagcttgccaacaattgtaaattgtttcaagagtcttggctaattgatcaatccccatagtatgtgatatgtcctgatcgatatcttctggatagagatgaactcctttttccaggattgaagctcccaatgcaatagacatatacaacatttcaattccattataatggtcagataaaccaactggtaatgaaaaacattggtttaaagttcgtaacattcttaggttatgtgcttctggcaaagcaggatgtccatcagggctatgttcaataataattttttcacatccggcactttttgctgtttcaactgctttgcatatatcagttaaggaagatcttccagtgtcaataatgagcggtaatcctgtttttgccagatgacgaattaaaggaatatggacaatgttcgatgaagaggttttgagtgcgcaggccccctgctttatcgcaaaatcaatagtgtccaattcataaatcgaaactatgaagggtaaattggcattctttaatagtgaaaatagcatactataatgttctaaaggcagtacttttctctcaattaatgagcggtagttttccttgcgaacctcacctgatttactggtataggtttcttcataatcaacaggtaggcaaatgtttgggttatttaaaatttctgtttttagtataagaggttggtatggtactttatccctgatggttataattttgtgtagcaattgttcagctatgccaagatcctgattaaaaaaagtacctatttctgctaaaaaaataggaggatgattttcttcaacagagaatgga

SEQ ID NO: 46 - DNA, 1060 bp, Legionella pneumophila; identical to SEQ ID NO: 43
  source 1..1060: mol_type = other DNA

SEQ ID NO: 47 - DNA, 1113 bp, Legionella pneumophila
  source 1..1113: mol_type = other DNA
gatagtcaatgagtcagccttttacaattggtccattctctgttgaagaaaatcatcctcctatttttttagcagaaataggtactttttttaatcaggatcttggcatagctgaacaattgctacacaaaattataaccatcagggataaagtaccataccaacctcttatactaaaaacagaaattttaaataacccaaacatttgcctacctgttgattatgaagaaacctataccagtaaatccggtgaggtccgcaaggaaaactaccgctcattaattgagagaaaagtactgcctttagaacattatagtatgctattttcactattaaagaatgccaatttacccttcatagtttcgatttatgaattggacactattgattttgcgataaagcagggtgcctgcgcactcaaaacctcttcatcgaacattgtccatgttcctttaattcgtcatctggcaaaaacaggattaccgctcattattgacactggaagatcttctttaactgatatatgcaaagcagttgaaacagcaaaaagtgccggatgtgaaaaaattattattgaacatagtcctgatggacatcctgctttgccagaagcacataacctaagaatgttacgaactttaaaccaatgtttttcattaccagttggtttatctgaccattataatggaattgaaatgttgtatatgtctattgcattgggagcttcaatcctggaaaaaggagttcatctctatccagaagatattgatcaggacatatcacatactatggggattgagcaattagccaagactcttgaaacaatttacaattgttggcaagctttaggtcaaccgatgcgagatctagaagctcctataaaaggagtacttggcagctctcaaagaacttgtttggttgcaaagcaaaaacttcaacctggctctctaattagccttgataatacaacctttgcctttccttgtaagggagtcccgacccaatactgggatctggtaaatggctgggaattacaaaacaaggtatctataggtgaacccattaaatggtcggatattaagcaaaaaaatgattgttagaaaccctacttgg

SEQ ID NO: 48 - DNA, 1113 bp, Legionella pneumophila; identical to SEQ ID NO: 47
  source 1..1113: mol_type = other DNA

(19) United States
(12) Patent Application Publication
(10) Pub. No.: US 2025/0290159 A1
(43) Pub. Date: Sep. 18, 2025

(54) METHOD FOR THE DETECTION OF LEGIONELLA

(71) Applicant: NATIONAL UNIVERSITY OF IRELAND, GALWAY, Galway (IE)

(72) Inventors: Thomas BARRY, Galway (IE); Kate REDDINGTON, Westport (IE); Elizabeth MINOGUE, Whitegate (IE)

(21) Appl. No.: 18/921,607

(22) Filed: Oct. 21, 2024

Related U.S. Application Data
(63) Continuation of application No. 16/339,968, filed on Apr. 5, 2019, now Pat. No. 12,123,061.

(30) Foreign Application Priority Data
Oct. 5, 2016 (EP) 16192489.9

(51) Int. Cl. C12Q 1/689

(52) U.S. Cl. CPC C12Q 1/689; C12Q 2600/112; C12Q 2600/16; Y02A 50/30

(57) ABSTRACT

This invention relates to the detection of Legionella, more specifically Legionella pneumophila, more specifically Legionella pneumophila serogroup 1, in a sample. Methods of detection, and assays and kits useful for detection, are also disclosed. The invention provides a method for identifying the presence, absence, or quantity of Legionella pneumophila serogroup 1 in a sample, the method comprising the steps of detecting the presence, absence, or quantity of at least part of the nucleic acid sequence of the lpg0768 gene or an expression product thereof, and correlating the presence, absence, or quantity of the at least part of the nucleic acid sequence of the lpg0768 gene or an expression product thereof, to the presence, absence, or quantity of Legionella pneumophila serogroup 1 in the sample.

### CROSS-REFERENCE TO RELATED APPLICATIONS

This application is a continuation of U.S. Ser. No. 16/339,968, filed Apr. 5, 2019, which is the U.S. National Stage of International Patent Application Serial No. PCT/EP2017/075265, filed Oct. 4, 2017, which claims the priority benefit of EP 16192489.9, filed Oct. 5, 2016, the contents of which are incorporated by reference in their entireties herein.

### SEQUENCE LISTING

The following application contains a sequence listing from the PCT application, submitted electronically as a ST.26 compliant XML file entitled “P118740US01 seqlist26.xml,” with a creation date of May 29, 2025, and a file size of 81,577 bytes in size, the contents of which are incorporated herein.

### FIELD OF THE INVENTION

This invention relates to the detection of Legionella, more specifically Legionella pneumophila, more specifically Legionella pneumophila serogroup 1, in a sample. Methods of detection, and assays and kits useful for detection are also disclosed.

### BACKGROUND OF THE INVENTION

Infections acquired in buildings, hospitals, and healthcare institutions affect approximately 2 million people, result in approximately 98,000 deaths, and cost a reported USD29 billion in the United States alone each year. The economic burden of infections acquired in hospitals in Europe is estimated to be €7 billion per annum.

Water, water distribution, and premise plumbing systems have been identified as a source of many of these infections, and can pose a significant threat to human health, especially in patients with weakened immune systems, and high dependency patients in critical care units. As infection control and prevention teams within clinical settings increasingly recognise the risks to patient health, a variety of testing and treatment technologies are being employed to attempt to eradicate pathogenic microorganisms from water. Despite the range of technologies being applied, the problem of waterborne infection, particularly from Legionella species, in the healthcare setting continues to persist.

Legionnaires' disease was first recognised in 1976 during an outbreak of pneumonia at an American Legion convention in Philadelphia. Legionella spp., the causative agent of Legionnaires disease, are responsible for outbreaks of both hospital acquired and community acquired pneumonia. In healthcare settings, there are increasing proportions of immunologically compromised patients leading to added potential for infection of patients by such opportunistic pathogenic microorganisms. The most common microorganism associated with Legionnaires disease is Legionella pneumophila. L. pneumophila serogroup 1 is the most clinically relevant. Currently, routine testing for L. pneumophila serogroup 1 is performed using traditional microbiological based techniques. This method is limited by the fastidious nature and long incubation periods required by Legionella spp. and also by the presence of viable but non-culturable Legionellae.

Prior art gold standard, culture-based methodologies are centuries old and are slow to deliver a result for Legionella, taking up to 2 weeks. Furthermore, prior art culture-based methodologies alone cannot specifically identify if L. pneumophila serogroup 1 is present in a sample. Serogroup 1 is the most important serogroup from a human health perspective, as it causes ˜95% of infections. There are also a number of prior art biochemical and immunodetection-based kits for these microorganisms. However, they are not very sensitive and are not truly quantitative.

### SUMMARY OF THE INVENTION

According to the present invention, there is provided a method for identifying the presence, absence, or quantity of Legionella pneumophila serogroup 1 in a sample, the method comprising the steps of:
(a) detecting the presence, absence, or quantity of at least part of the nucleic acid sequence of the lpg0768 gene, or an expression product thereof; and
(b) correlating the presence, absence, or quantity of the at least part of the nucleic acid sequence of the lpg0768 gene or expression product thereof to the presence, absence, or quantity of Legionella pneumophila serogroup 1 in the sample.

Optionally, the method further comprises the steps of:
(ai) detecting the presence, absence, or quantity of at least part of the nucleic acid sequence of the smpB gene, or an expression product thereof; and
correlating the presence, absence, or quantity of the at least part of the nucleic acid sequence of the smpB gene or expression product thereof to the presence, absence, or quantity of Legionella pneumophila in the sample.

Optionally or additionally, the method further comprises the steps of:
(aii) detecting the presence, absence, or quantity of at least part of the nucleic acid sequence of the ssrA gene, or an expression product thereof; and
correlating the presence, absence, or quantity of the at least part of the nucleic acid sequence of the ssrA gene or expression product thereof to the presence, absence, or quantity of Legionella genus in the sample.

Optionally, the method comprises the steps of:
(a) detecting the presence, absence, or quantity of at least part of the nucleic acid sequence of the lpg0768 gene or an expression product thereof;
(ai) detecting the presence, absence, or quantity of at least part of the nucleic acid sequence of the smpB gene or an expression product thereof;
(b) correlating the presence, absence, or quantity of the at least part of the nucleic acid sequence of the lpg0768 gene or expression product thereof to the presence, absence, or quantity of Legionella pneumophila serogroup 1 in the sample; and correlating the presence, absence, or quantity of the at least part of the nucleic acid sequence of the smpB gene or expression product thereof to the presence, absence, or quantity of Legionella pneumophila in the sample.

Optionally, the method comprises the steps of:
(a) detecting the presence, absence, or quantity of at least part of the nucleic acid sequence of the lpg0768 gene or an expression product thereof;
(aii) detecting the presence, absence, or quantity of at least part of the nucleic acid sequence of the ssrA gene or an expression product thereof;
(b) correlating the presence, absence, or quantity of the at least part the lpg0768 gene or expression product thereof to the presence, absence, or quantity of Legionella pneumophila serogroup 1 in the sample; and correlating the presence, absence, or quantity of the at least part of the nucleic acid sequence of the ssrA gene or expression product thereof to the presence, absence, or quantity of Legionella genus in the sample.

Optionally, the method comprising the steps of:
(a) detecting the presence, absence, or quantity of at least part of the nucleic acid sequence of the lpg0768 gene or an expression product thereof;
(ai detecting the presence, absence, or quantity of at least part of the nucleic acid sequence of the smpB gene or an expression product thereof;
(aii) detecting the presence, absence, or quantity of at least part of the nucleic acid sequence of the ssrA gene or an expression product thereof;
(b) correlating the presence, absence, or quantity of the at least part of the nucleic acid sequence the lpg0768 gene or expression product thereof to the presence, absence, or quantity of Legionella pneumophila serogroup 1 in the sample; correlating the presence, absence, or quantity of the at least part of the nucleic acid sequence of the smpB gene or expression product thereof to the presence, absence, or quantity of Legionella pneumophila in the sample; and correlating the presence, absence, or quantity of the at least part of the nucleic acid sequence of the ssrA gene or expression product thereof to the presence, absence, or quantity of Legionella genus in the sample.

Optionally, the presence of the at least part of the nucleic acid sequence of the lpg0768 gene or an expression product thereof indicates the presence of Legionella pneumophila serogroup 1.

Optionally, the absence of the at least part of the nucleic acid sequence of the lpg0768 gene or an expression product thereof indicates the absence of Legionella pneumophila serogroup 1.

Optionally, the quantity of the at least part of the nucleic acid sequence of the lpg0768 gene or an expression product thereof indicates the quantity of Legionella pneumophila serogroup 1.

Optionally, the at least part of the nucleic acid sequence of the lpg0768 gene comprises at least part of the nucleic acid sequence which encodes spore coat polysaccharide biosynthesis protein E (neuB).

Optionally, the at least part of the nucleic acid sequence of the lpg0768 gene comprises at least part of the nucleic acid sequence defined by Genbank Accession Number AE017354; base pairs 839972 to 840171.

Optionally, the at least part of the nucleic acid sequence of the lpg0768 gene comprises a nucleic acid sequence having at least 85% sequence identity with at least part of the nucleic acid sequence defined by any one of SEQ ID NOs 1 and 21-48.

Optionally, the at least part of the nucleic acid sequence of the lpg0768 gene comprises a nucleic acid sequence having at least 85%, 87.5%, 88%, 88.5%, 94%, 94.5%, 95%, or 100% sequence identity with at least part of the nucleic acid sequence defined by any one of SEQ ID NOs 1 and 21-48.

Optionally, the at least part of the nucleic acid sequence of the lpg0768 gene comprises a nucleic acid sequence defined by any one of SEQ ID NOs 1 and 21-48.

Optionally, the detecting step (a) comprises: contacting the sample with at least one primer capable of hybridizing to at least part of the at least part of the nucleic acid sequence of the lpg0768 gene, optionally at least part of the nucleic acid sequence defined by any one of SEQ ID NOs 1 and 21-48; under conditions suitable for an in vitro amplification, optionally an in vitro amplification selected from the group comprising: polymerase chain reaction (PCR), Nucleic Acid Sequence Based Amplification (NASBA), Rolling Circle Amplification (RCA), Ligase Chain Reaction (LCR), Signal Mediated Amplification of RNA Technology (SMART), Strand Displacement Amplification (SDA), Loop Mediated Isothermal Amplification (LAMP), Isothermal Multiple Displacement Amplification (IMDA), Helicase-Dependent Amplification (HDA), Single Primer Isothermal Amplification (SIPA), circular Helicase Dependent Amplification (cHDA), and Next Generation Sequencing (NGS).

Optionally, polymerase chain reaction (PCR) is selected from real-time PCR and quantitative real-time PCR.

Optionally, the polymerase chain reaction (PCR), optionally the real-time PCR, optionally the quantitative real-time PCR, further comprises pyrosequencing.

Optionally, the detecting step (a) comprises: contacting the sample with at least one primer having a nucleic acid sequence defined by at least one of SEQ ID NO. 2, SEQ ID NO. 3, and the reverse complement each thereof; under conditions suitable for an in vitro amplification, optionally an in vitro amplification selected from the group comprising: polymerase chain reaction (PCR), Nucleic Acid Sequence Based Amplification (NASBA), Rolling Circle Amplification (RCA), Ligase Chain Reaction (LCR), Signal Mediated Amplification of RNA Technology (SMART), Strand Displacement Amplification (SDA), Loop Mediated Isothermal Amplification (LAMP), Isothermal Multiple Displacement Amplification (IMDA), Helicase-Dependent Amplification (HDA), Single Primer Isothermal Amplification (SIPA), circular Helicase Dependent Amplification (cHDA), and Next Generation Sequencing (NGS).

Optionally, polymerase chain reaction (PCR) is selected from real-time PCR and quantitative real-time PCR.

Optionally, the polymerase chain reaction (PCR), optionally the real-time PCR, optionally the quantitative real-time PCR, further comprises pyrosequencing.

Optionally, the at least one primer has a final concentration of about 0.05-0.5 μM, optionally 0.05 μM, optionally 0.5 μM.

Optionally, the detecting step (a) comprises: contacting the sample with at least one probe capable of hybridizing to at least part of the at least part of the lpg0768 gene, optionally at least part of the nucleic acid sequence defined by any one of SEQ ID NOs 1 and 21-48, under conditions suitable for hybridization.

Optionally, the detecting step (a) comprises: contacting the sample with at least one probe having a nucleic acid sequence defined by at least one of SEQ ID NO. 4 and the reverse complement thereof, under conditions suitable for hybridisation.

Optionally, the at least one probe comprises at least one marker, optionally at least one dye, optionally at least one fluorophore, optionally hexachlorofluorescein.

Optionally or additionally, the at least one probe comprises at least one fluorescence quenching dye.

Optionally the polymerase chain reaction, or real-time PCR, or quantitative real-time PCR, cycling parameters comprise about 10 minutes incubation at about 95° C., optionally or additionally about 50 cycles of about 95° C. for about 10 seconds and about 60° C. for about 30 seconds, optionally or additionally followed by a cooling step at about 40° C. for about 10 seconds.

Optionally, the at least one probe has a final concentration of about 0.02-0.8 μM, optionally 0.02 μM, optionally 0.08 μM, optionally 0.2 μM, optionally 0.8 μM.

Optionally, the presence of the at least part of the nucleic acid sequence of the smpB gene or expression product thereof indicates the presence of Legionella pneumophila.

Optionally, the absence of the at least part of the nucleic acid sequence of the smpB gene or expression product thereof indicates the absence of Legionella pneumophila.

Optionally, the presence of the at least part of the nucleic acid sequence of the smpB gene or expression product thereof, and the presence of the at least part of the nucleic acid sequence of the lpg0768 gene or expression product thereof, indicates the presence of each of Legionella pneumophila serogroup 1 and Legionella pneumophila.

Optionally, the presence of the at least part of the nucleic acid sequence of the smpB gene or expression product thereof, and the absence of the at least part of the nucleic acid sequence of the lpg0768 gene or expression product thereof, indicates the presence of Legionella pneumophila and the absence of Legionella pneumophila serogroup 1.

Optionally, the presence of the at least part of the nucleic acid sequence of the smpB gene or expression product thereof, and the absence of the at least part of the nucleic acid sequence of the lpg0768 gene or an expression product thereof, indicates the presence of at least one organism selected from Legionella pneumophila serogroups 2-16.

Optionally, the absence of the at least part of the nucleic acid sequence of the smpB gene or expression product thereof, and the presence of the at least part of the lpg0768 gene, or expression product thereof, indicates at least one false result selected from: a false negative result in detecting step (ai), and a false positive result in detecting step (a).

Optionally, the absence of the at least part of the nucleic acid sequence of the smpB gene or expression product thereof, and the absence of the at least part of the nucleic acid sequence of the lpg0768 gene or expression product thereof, indicates the absence of each of Legionella pneumophila and Legionella pneumophila serogroup 1.

Optionally, the at least part of the nucleic acid sequence of the smpB gene comprises at least part of the nucleic acid sequence which encodes small protein B (smpB).

Optionally, the at least part of the nucleic acid sequence of the smpB gene comprises at least part of the nucleic acid sequence defined by Genbank Accession number AE017354; base pairs 3213961 to 3214076.

Optionally, the at least part of the nucleic acid sequence of the smpB gene comprises at least part of the nucleic acid sequence defined by SEQ ID NO. 6.

Optionally, the detecting step (ai) comprises: contacting the sample with at least one primer capable of hybridizing to at least part of the nucleic acid sequence of the smpB gene, optionally at least part of the nucleic acid sequence defined by SEQ ID NO. 6, under conditions suitable for an in vitro amplification, optionally an in vitro amplification selected from the group comprising: polymerase chain reaction (PCR), Nucleic Acid Sequence Based Amplification (NASBA), Rolling Circle Amplification (RCA), Ligase Chain Reaction (LCR), Signal Mediated Amplification of RNA Technology (SMART), Strand Displacement Amplification (SDA), Loop Mediated Isothermal Amplification (LAMP), Isothermal Multiple Displacement Amplification (IMDA), Helicase-Dependent Amplification (HDA), Single Primer Isothermal Amplification (SIPA), circular Helicase Dependent Amplification (cHDA), and Next Generation Sequencing (NGS).

Optionally, polymerase chain reaction (PCR) is selected from real-time PCR and quantitative real-time PCR.

Optionally, the polymerase chain reaction (PCR), optionally the real-time PCR, optionally the quantitative real-time PCR, further comprises pyrosequencing.

Optionally, the detecting step (ai) comprises: contacting the sample with at least one primer having a nucleic acid sequence defined by at least one of SEQ ID NO. 7, SEQ ID NO. 8, SEQ ID NO. 13, and the reverse complement each thereof, under conditions suitable for an in vitro amplification, optionally an in vitro amplification selected from the group comprising: polymerase chain reaction (PCR), Nucleic Acid Sequence Based Amplification (NASBA), Rolling Circle Amplification (RCA), Ligase Chain Reaction (LCR), Signal Mediated Amplification of RNA Technology (SMART), Strand Displacement Amplification (SDA), Loop Mediated Isothermal Amplification (LAMP), Isothermal Multiple Displacement Amplification (IMDA), Helicase-Dependent Amplification (HDA), Single Primer Isothermal Amplification (SIPA), circular Helicase Dependent Amplification (cHDA), and Next Generation Sequencing (NGS).

Optionally, polymerase chain reaction (PCR) is selected from real-time PCR and quantitative real-time PCR.

Optionally, the polymerase chain reaction (PCR), optionally the real-time PCR, optionally the quantitative real-time PCR, further comprises pyrosequencing.

Optionally, the at least one primer has a final concentration of about 0.05-0.5 μM, optionally 0.05 μM, optionally 0.5 μM.

Optionally the polymerase chain reaction, or real-time PCR, or quantitative real-time PCR, cycling parameters comprise about 5 minutes incubation at about 95° C., optionally or additionally 50 cycles at about 95° C. for about 10 seconds and about 60° C. for about 30 seconds, optionally or additionally followed by a cooling step at about 40° C. for about 10 seconds.

Optionally, the detecting step (ai) comprises: contacting the sample with at least one probe capable of hybridizing to at least part of the nucleic acid sequence of the smpB gene, optionally at least part of the nucleic acid sequence defined by SEQ ID NO. 6, under conditions suitable for hybridisation.

Optionally, the detecting step (ai) comprises: contacting the sample with at least one probe having a nucleic acid sequence defined by at least one of SEQ ID NO. 9 or the reverse complement thereof, under conditions suitable for hybridisation.

Optionally, the detecting step (ai) comprises: contacting the sample with at least one probe having a nucleic acid sequence defined by at least one of SEQ ID NO. 9, SEQ ID NO 51, SEQ ID NO. 52, and the reverse complement each thereof, under conditions suitable for hybridisation.

Optionally, the at least one probe comprises at least one marker, optionally at least one dye, optionally at least one fluorophore, optionally rhodamine.

Optionally, or additionally, the at least one probe comprises at least one fluorescence quenching dye.

Optionally, the at least one probe has a final concentration of about 0.02-0.8 μM, optionally 0.02 μM, optionally 0.08 μM, optionally 0.2 μM, optionally 0.8 μM.

Optionally, the presence of the at least part of the nucleic acid sequence of the ssrA gene or expression product thereof indicates the presence of Legionella genus.

Optionally, the absence of the at least part of the nucleic acid sequence of the ssrA gene or expression product thereof indicates the absence of Legionella genus.

Optionally, the presence of the at least part of the nucleic acid sequence of the ssrA gene or expression product thereof, and the presence of the at least part of the lpg0768 gene or expression product thereof, indicates the presence of each of the Legionella genus and Legionella pneumophila serogroup 1.

Optionally, the presence of the at least part of the nucleic acid sequence of the ssrA gene or expression product thereof, and the absence of the at least part of the nucleic acid sequence of the lpg0768 gene or expression product thereof, indicates the presence of the Legionella genus and the absence of Legionella pneumophila serogroup 1.

Optionally, the presence of the at least part of the nucleic acid sequence of the ssrA gene or expression product thereof, and the absence of the at least part of the nucleic acid sequence of the lpg0768 gene or expression product thereof, indicates the presence of at least one organism selected from Legionella pneumophila serogroups 2-16, Legionella anisa, L. birminghamensis, L. birminghamensis, L. bozemanii, L. bozemanii, L. cincinnatiensis, L. dumoffii, L. dumoffii, L. erythra, L. feeleii-1, L. feelii-2, L. gormanii, L. gormanii, L. hackeliae-1, L. hackeliae-2, L. jordanis, L. jordanis, L. lansingensis, L. longbeachae-1, L. maceachemii, L. miodadei, L. oakridgensis, L. oakridgensis, L. parisiensis, L. wadworthii, L. cherrii, L. jamestowniensis, L. londoniensis, L. taurinsis, L. moravica, L. adelaidensis, L. donaldsonii, L. gratiana, L. gresilensis, L. fairfieldensis, L. israelensis, L. fallonii, L. brunensis, L. busanensis, L. quinlivanii, and L. rubrilicens or other non-Legionella pneumophila species

Optionally, the absence of the at least part of the nucleic acid sequence of the ssrA gene or expression product thereof, and the presence of the at least part of the nucleic acid sequence of the lpg0768 gene or expression product thereof, indicates at least one false result selected from: a false negative result in detecting step (aii), and a false positive result in detecting step (a).

Optionally, the absence of the at least part of the nucleic acid sequence of the ssrA gene or expression product thereof, and the absence of the at least part of the nucleic acid sequence of the lpg0768 gene or expression product thereof, indicates the absence of each of the Legionella genus and Legionella pneumophila serogroup 1.

Optionally, the presence of the at least part of the nucleic acid sequence of the ssrA gene or expression product thereof, and the presence of the at least part of the nucleic acid sequence of the smpB gene or expression product thereof, and the presence of the at least part of the nucleic acid sequence of the lpg0768 gene or expression product thereof, indicates the presence of each of the Legionella genus, Legionella pneumophila, and Legionella pneumophila serogroup 1.

Optionally, the presence of the at least part of the nucleic acid sequence of the ssrA gene or expression product thereof, and the presence of the at least part of the nucleic acid sequence of the smpB gene or expression product thereof, and the absence of the at least part of the nucleic acid sequence of the lpg0768 gene or an expression product thereof, indicates the presence of Legionella genus and Legionella pneumophila, and the absence of Legionella pneumophila serogroup 1.

Optionally, the presence of the at least part of the nucleic acid sequence of the ssrA gene or expression product thereof, and the presence of the at least part of the nucleic acid sequence of the smpB gene or expression product thereof, and the absence of the at least part of the nucleic acid sequence of the lpg0768 gene or expression product thereof, indicates the presence of the Legionella genus and at least one organism selected from Legionella pneumophila serogroup 2-16.

Optionally, the presence of the at least part of the nucleic acid sequence of the ssrA gene or expression product thereof, and the absence of the at least part of the nucleic acid sequence of the smpB gene or expression product thereof, and the presence of the at least part of the lpg0768 gene or expression product thereof, indicates the presence of the Legionella genus, and also indicates at least one false result selected from: a false negative result in detecting step (ai), a false positive result in detecting step (a).

Optionally, the presence of the at least part of the nucleic acid sequence of the ssrA gene or expression product thereof, and the absence of the at least part of the nucleic acid sequence of the smpB gene or expression product thereof, and the presence of the at least part of the nucleic acid sequence of the lpg0768 gene or expression product thereof, indicates the presence of at least one organism selected from Legionella anisa, L. birminghamensis, L. birminghamensis, L. bozemanii, L. bozemanii, L. cincinnatiensis, L. dumoffii, L. dumoffii, L. erythra, L. feeleii-1, L. feelii-2, L. gormanii, L. gormanii, L. hackeliae-1, L. hackeliae-2, L. jordanis, L. jordanis, L. lansingensis, L. longbeachae-1, L. maceachemii, L. miodadei, L. oakridgensis, L. oakridgensis, L. parisiensis, L. wadworthii, L. cherrii, L. jamestowniensis, L. londoniensis, L. taurinsis, L. moravica, L. adelaidensis, L. donaldsonii, L. gratiana, L. gresilensis, L. fairfieldensis, L. israelensis, L. fallonii, L. brunensis, L. busanensis, L. quinlivanii, L. rubrilicens, or other non-Legionella pneumophila species, and also indicates at least one false result selected from: a false negative result in detecting step (ai), a false positive result in detecting step (a).

Optionally, the presence of the at least part of the nucleic acid sequence of the ssrA gene or expression product thereof, and the absence of the at least part of the nucleic acid sequence of the smpB gene or expression product thereof, and the absence of the at least part of the nucleic acid sequence of the lpg0768 gene or expression product thereof, indicates the presence of at least one organism selected from the Legionella genus, and the absence of each of Legionella pneumophila species and Legionella pneumophila serogroup 1.

Optionally, the presence of the at least part of the nucleic acid sequence of the ssrA gene or expression product thereof, and the absence of the at least part of the nucleic acid sequence of the smpB gene or expression product thereof, and the absence of the at least part of the nucleic acid sequence of the lpg0768 gene or expression product thereof, indicates the presence of at least one organism selected from Legionella anisa, L. birminghamensis, L. birminghamensis, L. bozemanii, L. bozemanii, L. cincinnatiensis, L. dumoffii, L. dumoffii, L. erythra, L. feeleii-1, L. feelii-2, L. gormanii, L. gormanii, L. hackeliae-1, L. hackeliae-2, L. jordanis, L. jordanis, L. lansingensis, L. longbeachae-1, L. maceachemii, L. miodadei, L. oakridgensis, L. oakridgensis, L. parisiensis, L. wadworthii, L. cherrii, L. jamestowniensis, L. londoniensis, L. taurinsis, L. moravica, L. adelaidensis, L. donaldsonii, L. gratiana, L. gresilensis, L. fairfieldensis, L. israelensis, L. fallonii, L. brunensis, L. busanensis, L. quinlivanii, L. rubrilicens, or other non-Legionella pneumophila species, and the absence of Legionella pneumophila species or Legionella pneumophila serogroup 1.

Optionally, the absence of the at least part of the nucleic acid sequence of the ssrA gene or expression product thereof, the presence of the at least part of the nucleic acid sequence of the smpB gene or expression product thereof, and the presence of the at least part of the nucleic acid sequence of the lpg0768 gene or expression product thereof, indicates at least one false result selected from: a false negative result in detecting step (aii), a false positive result in detecting step (ai), and a false positive result in detecting step (a).

Optionally, the absence of the at least part of the nucleic acid sequence of the ssrA gene or expression product thereof, the presence of the at least part of the nucleic acid sequence of the smpB gene or expression product thereof, and the absence of the at least part of the nucleic acid sequence of the lpg0768 gene or expression product thereof, indicates at least one false result selected from: a false negative result in detecting step (aii), a false positive result in detecting step (ai), and a false negative result in detecting step (a).

Optionally, the absence of the at least part of the nucleic acid sequence of the ssrA gene or expression product thereof, the absence of the at least part of the nucleic acid sequence of the smpB gene or expression product thereof, and the absence of the at least part of the nucleic acid sequence of the lpg0768 gene or expression product thereof, indicates the absence of each of the Legionella genus, Legionella pneumophila species, and Legionella pneumophila serogroup 1.

Optionally, the at least part of the nucleic acid sequence of the ssrA gene comprises at least part of the nucleic acid sequence defined by Genbank Accession number AE017354; base pairs 172917 to 173144.

Optionally, the at least part of the nucleic acid sequence of the ssrA gene comprises at least part of the nucleic acid sequence defined by SEQ ID NO. 5.

Optionally, the detecting step (aii) comprises: contacting the sample with at least one primer capable of hybridizing to at least part of the nucleic acid sequence of the ssrA gene, optionally at least part of the nucleic acid sequence defined by SEQ ID NO. 5, under conditions suitable for an in vitro amplification, optionally an in vitro amplification selected from the group comprising: polymerase chain reaction (PCR), Nucleic Acid Sequence Based Amplification (NASBA), Rolling Circle Amplification (RCA), Ligase Chain Reaction (LCR), Signal Mediated Amplification of RNA Technology (SMART), Strand Displacement Amplification (SDA), Loop Mediated Isothermal Amplification (LAMP), Isothermal Multiple Displacement Amplification (IMDA), Helicase-Dependent Amplification (HDA), Single Primer Isothermal Amplification (SIPA), circular Helicase Dependent Amplification (cHDA), and Next Generation Sequencing (NGS).

Optionally, polymerase chain reaction (PCR) is selected from real-time PCR and quantitative real-time PCR.

Optionally, the polymerase chain reaction (PCR), optionally the real-time PCR, optionally the quantitative real-time PCR, further comprises pyrosequencing.

Optionally, the detecting step (aii) comprises: contacting the sample with at least one primer having a nucleic acid sequence defined by at least one of SEQ ID NO. 10, SEQ ID NO. 11, and the reverse complement each thereof, under conditions suitable for an in vitro amplification, optionally an in vitro amplification selected from the group comprising: polymerase chain reaction (PCR), Nucleic Acid Sequence Based Amplification (NASBA), Rolling Circle Amplification (RCA), Ligase Chain Reaction (LCR), Signal Mediated Amplification of RNA Technology (SMART), Strand Displacement Amplification (SDA), Loop Mediated Isothermal Amplification (LAMP), Isothermal Multiple Displacement Amplification (IMDA), Helicase-Dependent Amplification (HDA), Single Primer Isothermal Amplification (SIPA), circular Helicase Dependent Amplification (cHDA), and Next Generation Sequencing (NGS).

Optionally, polymerase chain reaction (PCR) is selected from real-time PCR and quantitative real-time PCR.

Optionally, the polymerase chain reaction (PCR), optionally the real-time PCR, optionally the quantitative real-time PCR, further comprises pyrosequencing.

Optionally, the at least one primer has a final concentration of about 0.05-0.5 μM, optionally 0.05 μM, optionally 0.5 μM.

Optionally the polymerase chain reaction, or real-time PCR, or quantitative real-time PCR, cycling parameters comprise about 10 minutes incubation at about 95° C., optionally or additionally 50 cycles at about 95° C. for about 10 seconds and about 63° C. for about 30 seconds, optionally or additionally followed by a cooling step at about 40° C. for about 10 seconds.

Optionally, the detecting step (ai) comprises: contacting the sample with a probe capable of hybridizing to at least part of the nucleic acid sequence of the ssrA gene, optionally at least part of the nucleic acid sequence defined by SEQ ID NO. 5, under conditions suitable for hybridisation.

Optionally, the detecting step (ai) comprises: contacting the sample with a probe having a nucleic acid sequence defined by SEQ ID NO. 12 or the reverse complement thereof, under conditions suitable for hybridisation.

Optionally, the at least one probe comprises at least one marker, optionally at least one dye, optionally at least one fluorophore, optionally fluorescein.

Optionally, or additionally, the at least one probe comprises at least one fluorescence quenching dye.

Optionally, the at least one probe has a final concentration of about 0.02-0.8 μM, optionally 0.02 μM, optionally 0.08 μM, optionally 0.2 μM, optionally 0.8 μM.

Optionally, the method is a multiplex method.

Optionally, the method is a multiplex polymerase chain reaction method.

This invention provides a multiplex in vitro nucleic acid amplification method for identifying a serogroup of Legionella pneumophila present in a sample, wherein the method comprises detecting the presence or absence of a plurality of nucleic acid molecule targets in the sample in one reaction, wherein at least one of the nucleic acid molecule targets is present in the genome of one or more, but not all, of the serogroups of Legionella pneumophila.

Previous methods for the detection of Legionella pneumophila have not been capable of specifically identifying Legionella pneumophila serogroup 1 in a manner that is practically useful. This means that diagnosis and treatment provision is not tailored to the specific serogroup present unless extensive experimentation is carried out. This requires significant time and effort that is incompatible with rapid and effective diagnosis and treatment. This invention, for the first time, provides a method that is able to identify members of Legionella pneumophila serogroup 1 in a rapid and easily-interpretable manner. The inventors have surprisingly found that there is sufficient variation between serogroups yet sufficient conservation between isolates of the same serogroup to specifically identify Legionella pneumophila serogroup 1 in a single reaction multiplex nucleic acid amplification assay. By identifying and characterising a series of sequences that are either shared or not shared between the different members of the Legionella, the present invention thus allows the use of multiplex nucleic acid amplification methods to specifically detect Legionella pneumophila serogroup 1.

The serogroups that make up Legionella pneumophila are closely related but differ significantly in their pathology and susceptibility to certain treatments. Therefore, although it is important to be able to distinguish between the different serogroups, it has previously not been possible to do this in any straightforward way that is amenable to use in rapid diagnosis. The different members of Legionella pneumophila share a significant proportion of their genetic material. The present invention has successfully identified sufficient differences between the genomes of the members of Legionella pneumophila to be able to distinguish between them in multiplex in vitro nucleic acid amplification assays. In contrast to the methods of the prior art, the methods of the present invention allow a single multiplex reaction to be performed that gives clear signals to identify which serogroup is present in the tested sample. It is not necessary to run gels or to undertake complex interpretation of the results.

The present inventors have surprisingly found that it is possible to identify serogroup 1 of Legionella pneumophila, despite the high sequence homology that exists between the serogroups of Legionella pneumophila. In particular, the present invention provides a multiplex in vitro nucleic acid amplification assay that is capable of identifying serogroups of Legionella pneumophila, such as Legionella pneumophila serogroup 1 in a single reaction. The inventors have also discovered that the presence of at least one organism selected from the other serogroups in the Legionella pneumophila species, including any of serogroups 1-16, can also be identified in the same single reaction. Furthermore, the inventors have devised a method for detecting the presence of other members of the Legionella genus, including Legionella anisa, L. birminghamensis, L. birminghamensis, L. bozemanii, L. bozemanii, L. cincinnatiensis, L. dumoffii, L. dumoffii, L. erythra, L. feeleii-1, L. feelii-2, L. gormanii, L. gormanii, L. hackeliae-1, L. hackeliae-2, L. jordanis, L. jordanis, L. lansingensis, L. longbeachae-1, L. maceachemii, L. miodadei, L. oakridgensis, L. oakridgensis, L. parisiensis, L. wadworthii, L. cherrii, L. jamestowniensis, L. londoniensis, L. taurinsis, L. moravica, L. adelaidensis, L. donaldsonii, L. gratiana, L. gresilensis, L. fairfieldensis, L. israelensis, L. fallonii, L. brunensis, L. busanensis, L. quinlivanii, and L. rubrilicens.

The present invention contemplates the use of any appropriate method for amplification of target regions. The method of amplification will in general be PCR. Real-time PCR (RT-PCR) is one example of this. In certain embodiments RT-PCR using fluorescent reporter probes is used. The use of the reporter probes significantly increases specificity, and enables quantification even in the presence of non-specific DNA amplification. Fluorescent probes are particularly suited to multiplex assays, as specific probes with different-coloured labels can be used. RT-PCR relies on a DNA-based probe with a fluorescent reporter at one end and a quencher of fluorescence at the opposite end of the probe. The close proximity of the reporter to the quencher prevents detection of its fluorescence; breakdown of the probe by the 5′ to 3′ exonuclease activity of the DNA polymerase (for example Taq polymerase) breaks the reporter-quencher proximity, and results in emission of fluorescence. An increase in the product targeted by the reporter probe at each PCR cycle therefore causes a proportional increase in fluorescence due to the breakdown of the probe and release of the reporter.

Further, the methods of the invention may therefore be configured to allow quantitation, i.e. the method comprises determining the amount of a member of a group of bacterial organisms in a sample. This may be achieved e.g. by selecting appropriate parameters for amplification (e.g. the number of cycles of amplification).

Other appropriate methods of amplification are referred to below.

Also contemplated for use in the present invention is Nucleic Acid Sequence Based Amplification (NASBA). Nucleic acid sequence-based amplification (NASBA) is an isothermal amplification technique which uses three enzymes (RNase H, AMV reverse transcriptase and T7 RNA polymerase) working in concert at a low isothermal temperature (generally 41° C.). The product of a NASBA reaction is mainly single-stranded RNA, which can be detected by gel electrophoresis, enzyme-linked gel assay (ELGA) or electrochemiluminescent detection (ECL). Alternatively, NASBA products can be detected in real time using molecular beacons included in the reaction. In microbial diagnostics, NASBA has been successfully combined with electrochemiluminescent (ECL), ELISA labelled dendrimer and molecular beacon-based methods to detect and identify viral and bacterial pathogens (Scheler et al., 2009).

Also contemplated for use in the present invention is Rolling Circle Amplification (RCA). RCA describes a process of unidirectional nucleic acid replication that can rapidly synthesize multiple copies of circular molecules of DNA or RNA. RCA is a technology that is adaptable to an on-chip signal amplification format. RCA is well suited to solid phase formats such as microarrays for generating localized signals at specific microarray locations. This distinctive property of RCA should allow many assays to be performed simultaneously (multiplexing) without interference.

Also contemplated for use in the present invention is the Ligase Chain Reaction (LCR). LCR uses two complementary pairs of probes which, when the correct template is available, hybridize next to each other and then are ligated together. These ligated probes plus the original template serve as the template for the next cycle of hybridization and ligation. As subsequent cycles are performed, the amplification proceeds exponentially. A commercially available kit using this technology is the LCx M. tuberculosis complex specific kit available from Abbott Diagnostics.

Further isothermal amplification technologies that are contemplated for use with the present invention are provided in and include signal mediated amplification of RNA technology (SMART), strand displacement amplification (SDA), loop mediated isothermal amplification (LAMP), isothermal multiple displacement amplification (IMDA), helicase-dependent amplification (HDA), single primer isothermal amplification (SIPA) and circular helicase dependent amplification (cHDA). As exemplified by SMART, the amplification method used with the invention may comprise signal amplification rather than target amplification.

Also contemplated for use in the present invention is Next Generation Sequencing (NGS). Next generation sequencing is a relatively new field of sequencing which allows for the rapid high throughput process. NGS has the capacity to generate gigabases of nucleotide sequence, depending on the instrument used, in a single run. A recently described assay combines the use of real-time PCR in combination with pyrosequencing which allows for the rapid detection of MTC DNA in addition to sequencing of an 81-bp core region of the rpoB gene associated with rifampin resistance (Halse et al., 2010).

Optionally the sample substantially comprises a water sample.

Optionally the sample substantially comprises an environmental water sample.

Optionally the sample substantially comprises a sample from a water distribution system.

Optionally the sample substantially comprises a sample from a plumbing system.

Optionally the sample substantially comprises a sample from a healthcare facility water system.

Also disclosed is a Legionella detection kit comprising at least one primer or probe capable of hybridizing to at least one nucleic acid sequence selected from: at least part of the nucleic acid sequence of the lpg0768 gene or an expression product thereof, at least part of the nucleic acid sequence of the smpB gene or an expression product thereof, at least part of the nucleic acid sequence of the ssrA gene or an expression product thereof; and instructions for use.

Optionally, the kit comprises at least one primer or probe capable of hybridizing to at least one nucleic acid sequence defined by any of SEQ ID NOs 1, 5, 6, and 21-48, or any expression product each thereof; and instructions for use.

Optionally, the kit comprises at least one primer or probe having the nucleic acid sequence defined by any of SEQ ID NOs 2, 3, 4, 7, 8, 9 and 13; and instructions for use.

Also disclosed is a Legionella detection assay comprising a solid support and at least one primer or probe capable of hybridizing to at least one nucleic acid sequence selected from: at least part of the nucleic acid sequence of the lpg0768 gene or an expression product thereof, at least part of the nucleic acid sequence of the smpB gene or an expression product thereof, at least part of the nucleic acid sequence of the ssrA gene or an expression product thereof.

Optionally, the assay comprises a solid support and at least one primer or probe capable of hybridizing to at least one nucleic acid sequence defined by any of SEQ ID NOs 1, 5, 6, and 21-48, or any expression product each thereof.

Optionally, the assay comprises a solid support and at least one primer or probe having the nucleic acid sequence defined by any of SEQ ID NOs 2, 3, 4, 7, 8, 9 and 13.

Optionally, the assay is a microarray comprising at least one primer or probe capable of hybridizing to at least one nucleic acid sequence selected from: at least part of the nucleic acid sequence of the lpg0768 gene or an expression product thereof, the at least part of the nucleic acid sequence of the smpB gene or an expression product thereof, the at least part of the nucleic acid sequence of the ssrA gene or an expression product thereof.

Optionally, the microarray comprises at least one primer or probe capable of hybridizing to at least one nucleic acid sequence defined by any of SEQ ID NOs 1, 5, 6, and 21-48, or any expression product each thereof.

Optionally, the microarray discloses at least one primer or probe having the nucleic acid sequence defined by any of SEQ ID NOs 2, 3, 4, 7, 8, 9 and 13.

Also disclosed is a method for detecting Legionnaires' disease comprising a method according to the invention, wherein the presence of at least part of the nucleic acid sequence of the lpg0768 gene or expression product thereof indicates the presence of Legionnaires' disease.

Optionally, the method for detecting Legionnaires' disease comprises the steps of:
(a) detecting the presence, absence, or quantity of at least part of the nucleic acid sequence of the lpg0768 gene or an expression product thereof; and
(b) correlating the presence, absence, or quantity of the at least part of the nucleic acid sequence of the lpg0768 gene or expression product thereof, to the presence, absence, or quantity of Legionnaires' disease.

Optionally, the method is an in vitro method.

Optionally, any probe used in the method for detecting Legionnaires' disease may further comprise at least one dye, optionally at least one fluorophore, optionally hexachlorofluorescein, optionally rhodamine, optionally fluorescein.

Optionally or additionally, the probe may further comprise at least one fluorescence quenching dye.

The term “expression product” refers to any molecule that may be obtained from a nucleic acid sequence by transcription, translation, post-translational modification, or reverse transcription;

including but not limited to RNA, amino acid polypeptide, protein, and complementary DNA.

The term “sg1” is synonymous with the term “serogroup 1”.

The term “terminal quencher” refers to a fluorescence quenching dye, which can be located at or adjacent at least one end, optionally located at or adjacent at least one terminal end, of a probe; for example, located at or adjacent at least the 3′ end of an oligonucleotide probe.

The term “internal quencher” refers to a fluorescence quenching dye, which can be located between the ends, optionally located between the terminal ends of a probe; for example, located between the 3′ and 5′ ends of an oligonucleotide probe.

### BRIEF DESCRIPTION OF THE DRAWINGS

Embodiments of the invention will now be described with reference to the accompanying drawings in which:

FIG. 1(a) illustrates quantitative real-time PCR amplification curves, showing inclusivity testing of the L. pneumophila serogroup 1 assay targeting a region of the lpg0768 gene for detection of L. pneumophila serogroup 1 strains;

FIG. 1(b) illustrates quantitative real-time PCR amplification curves, showing exclusivity testing of the L. pneumophila serogroup 1 assay targeting a region of the lpg0768 gene. No cross reactions were observed with strains closely related to L. pneumophila serogroup 1;

FIG. 2(a) illustrates quantitative real-time PCR amplification curves, showing inclusivity testing of the L. pneumophila species assay targeting a region of the smpB gene for detection of L. pneumophila strains;

FIG. 2(b) illustrates quantitative real-time PCR amplification curves, showing exclusivity testing of the L. pneumophila species assay targeting a region of the smpB gene. No cross reactions were observed with non-Legionella pneumophila species and strains tested for.

FIG. 3(a) illustrates quantitative real-time PCR amplification curves, showing inclusivity testing of species of the Legionella genus assay targeting a region of the ssrA gene for detection of Legionella strains, using the multiplex assay;

FIG. 3(b) illustrates quantitative real-time PCR amplification curves, showing the specificity of the L. pneumophila serogroup 1 assay targeting a region of the lpg0768 gene for detection of L. pneumophila serogroup 1 isolates;

FIG. 3(c) illustrates quantitative real-time PCR amplification curves, showing the specificity of the L. pneumophila species assay targeting a region of the smpB gene for detection of all L. pneumophila strains and non-detection of other Legionella species; and

FIG. 3(d) illustrates quantitative real-time PCR amplification curves, showing detection of the Internal Amplification Control (IAC) assay in each sample tested for.

FIG. 4(A) illustrates quantitative real-time PCR amplification curves, showing inclusivity testing of species of the Legionella genus assay targeting a region of the ssrA gene for detection of Legionella strains, using the multiplex assay;

FIG. 4(B) illustrates quantitative real-time PCR amplification curves, showing the specificity of the L. pneumophila serogroup 1 assay targeting a region of the lpg0768 gene for detection of L. pneumophila serogroup 1 isolates;

FIG. 4(C) illustrates quantitative real-time PCR amplification curves, showing the specificity of the L. pneumophila species assay targeting a region of the smpB gene for detection of all L. pneumophila strains and non-detection of other Legionella species; and

FIG. 4(D) illustrates quantitative real-time PCR amplification curves, showing detection of the Internal Amplification Control (IAC) assay in each sample tested for.

### EXAMPLES

Embodiments of the present invention will now be described with reference to the following non-limiting examples:

### Example 1—Real-Time PCR Assay for the Detection of Legionella pneumophila Serogroup 1

### In Silico Target Identification

Publicly available whole genome sequence information was retrieved from the National Center for Biotechnology Information website (ncbi.nlm.nih.gov/) for Legionella pneumophila serogroup 1 and Legionella pneumophila serogroup 6 and serogroup 12. Regions potentially unique to L. pneumophila serogroup 1 were identified using WEBACT (webact.org/). This resulted in a number of regions of difference in L. pneumophila serogroup 1. Each identified putative target nucleotide sequence was BLAST analysed (blast.ncbi.nlm.nih.gov/Blast.cgi) to determine if they were present in all L. pneumophila serogroup 1 and absent in all other L. pneumophila serogroups or closely related Legionella species. One region was present in all L. pneumophila serogroup 1 analysed with very low or no sequence similarity to other L. pneumophila serogroups or closely related Legionella species. This region was the lpg0768 gene (SEQ ID NOs 1 and 21-48) which codes for spore coat polysaccharide biosynthesis protein E, NeuB. Closely related nucleotide sequences were aligned using ClustalW (ebi.ac.uk/Tools/msa/clustalw2/).

In this example, the following exemplary real-time PCR assay primers and probe for the lpg0768 gene (SEQ ID NOs 1 and 21-48) were used: spore coat polysaccharide biosynthesis protein E forward primer (SEQ ID NO. 2), spore coat polysaccharide biosynthesis protein E reverse primer (SEQ ID NO. 3), spore coat polysaccharide biosynthesis protein E probe (SEQ ID NO. 4). The spore coat probe (SEQ ID NO. 4) comprised at least one marker, optionally at least one dye, optionally at least one fluorophore, optionally hexachlorofluorescein (5′ HEX, Integrated DNA Technologies). Optionally, the spore coat polysaccharide biosynthesis protein E probe (SEQ ID NO. 4) comprises at least one fluorescence quenching dye, optionally an internal quencher, such as ZENT quencher, Integrated DNA Technologies; and/or a terminal quencher, such as 3′ Iowa Black® FQ, Integrated DNA Technologies; or Black Hole Quencher 1°, LGC Biosearch.

### Bacterial Strains, Culture Media, and Growth Conditions

A panel of 26 Legionella pneumophila strains and 41 other Legionella species were used in this study (Table 1(a) and 1(b)). These species and strains were purchased from the German Collection of Microorganisms and Cell Cultures (Deutsche Sammlung von Mikroorganismen und Zellkulturen GmbH, DSMZ. Braunschweig, Germany), the National Collection of Type Cultures (NCTC, Public Health England, Salisbury, United Kingdom), American Type Culture Collection (ATCC, provided by LGC standards, Middlesex United Kingdom). All Legionella species and strains were cultured on buffered charcoal yeast extract (BCYE) media at 37° C. 18-24 hours or until sufficient growth was observed.

TABLE 1(a)
Inclusivity panel

| | Culture |
|---|---|
| Strain | collection number |
| Legionella pneumophila subsp. pneumophila-1 | DSM 7513 |
| Legionella pneumophila subsp. pneumophila-1 | NCTC 11191 |
| Legionella pneumophila subsp. pneumophila-1 | NCTC 11231 |
| Legionella pneumophila subsp. pneumophila-1 | NCTC 11286 |
| Legionella pneumophila subsp. pneumophila-1 | NCTC 11378 |
| Legionella pneumophila subsp. pneumophila-1 | NCTC 11404 |
| Legionella pneumophila subsp. pneumophila-1 | DSM 25019 |
| Legionella pneumophila subsp. pneumophila-1 | DSM 25021 |
| Legionella pneumophila subsp. pneumophila-1 | DSM 25199 |
| Legionella pneumophila subsp. pneumophila-1 | DSM 25200 |
| Legionella pneumophila subsp. pneumophila-1 | DSM 25213 |
| Legionella pneumophila subsp. pneumophila-1 | DSM 37564 |

TABLE 1(b)
Exclusivity panel

| | Culture |
|---|---|
| Species | collection number |
| Legionella pneumophila subsp. fraseri-4 | DSM 7514 |
| Legionella pneumophila subsp. pasculei-5 | DSM 7515 |
| Legionella pneumophila subsp. pneumophila-1 | NCTC 11231 |
| Legionella pneumophila subsp. pneumophila-3 | NCTC 11232 |
| Legionella pneumophila subsp. pneumophila-6 | NCTC 11287 |
| Legionella pneumophila subsp. pneumophila-5 | NCTC 11417 |
| Legionella pneumophila subsp. pneumophila-7 | NCTC 11984 |
| Legionella pneumophila subsp. pneumophila-8 | NCTC 11985 |
| Legionella pneumophila subsp. pneumophila-9 | NCTC 11986 |
| Legionella pneumophila subsp. pneumophila-10 | NCTC 12000 |
| Legionella pneumophila subsp. pneumophila-14 | NCTC 12174 |
| Legionella pneumophila subsp. pneumophila-11 | NCTC 12179 |
| Legionella pneumophila subsp. pneumophila-12 | NCTC 12180 |
| Legionella pneumophila subsp. pneumophila-13 | NCTC 12181 |
| Legionella anisa | DSM 17627 |
| Legionella birminghamensis | DSM 19232 |
| Legionella birminghamensis | ATCC 700709 |
| Legionella bozemanii | NCTC 11975 |
| Legionella bozemanii | DSM 16523 |
| Legionella cincinnatiensis | DSM 19233 |
| Legionella dumoffii | DSM 17625 |
| Legionella dumoffii | ATCC 33343 |
| Legionella erythra | DSM 17644 |
| Legionella feeleii-1 | DSM 17645 |
| Legionella feelii-2 | NCTC 11978 |
| Legionella gormanii | DSM 16641 |
| Legionella gormanii | ATCC 43769 |
| Legionella hackeliae-1 | DSM 19214 |
| Legionella hackeliae-2 | NCTC 11980 |
| Legionella jordanis | DSM 19212 |
| Legionella jordanis | ATCC 700762 |
| Legionella lansingensis | DSM 19556 |
| Legionella longbeachae-1 | DSM 10572 |
| Legionella maceachemii | DSM 16642 |
| Legionella miodadei | NCTC 11403 |
| Legionella oakridgensis | NCTC 11531 |
| Legionella oakridgensis | ATCC 700515 |
| Legionella parisiensis | DSM 19216 |
| Legionella wadworthii | NCTC 11532 |
| Legionella cherrii | DSM 19213 |
| Legionella jamestowniensis | DSM 19215 |
| Legionella londoniensis | DSM 21234 |
| Legionella taurinsis | CCUG 44901 |
| Legionella moravica | DSM 19234 |
| Legionella adelaidensis | DSM 19888 |
| Legionella donaldsonii | ATCC BAA-693 |
| Legionella gratiana | DSM 21233 |
| Legionella gresilensis | DSM 21218 |
| Legionella fairfieldensis | NCTC 12488 |
| Legionella israelensis | DSM 19235 |
| Legionella fallonii | CCUG 43887 |
| Legionella brunensis | DSM 19236 |
| Legionella busanensis | ATCC BAA-518 |
| Legionella quinlivanii | NCTC 12434 |
| Legionella rubrilicens | DSM 11884 |

### Dna Isolation and Quantification

Genomic DNA from Legionella cultures were isolated using a modified procedure combining mechanical lysis (IDI lysis kit; Becton Dickenson, Canada) and purification using a DNA purification kit (quick gDNA kit; Zymo Research, Irvine, CA, USA). Briefly, a loop of culture was resuspended in 250 ul lysis buffer (IDI lysis buffer). The suspension was transferred to a cell lysis tube (GeneOhm) and bead beaten (Mini-Bead-Beater-16; Stratech, United Kingdom) for 3 minutes. After bead beating, 200 μl was transferred to a nucleic acid purification spin column (Zymo-Spin™ Column) in a collection tube and steps 2 to 5 of the procedure for purification of total DNA from cell suspensions were followed, according to the manufacturer's instructions.

### Assay Design

All oligonucleotide primers and probes used in this study were designed in accordance with published guidelines.

### Real-Time PCR

This Legionella pneumophila serogroup 1 specific assay was then tested in a real-time PCR format against a panel of well characterised Legionella species to determine assay specificity (Inclusivity and Exclusivity testing) and suitability of the target nucleotide sequence as a diagnostics marker.

Real-time PCR was performed on a real-time PCR amplification device (LightCycler® 480 Instrument, Roche Diagnostics) using a real-time PCR probes kit (LightCycler® 480 Probes Master kit, Roche Diagnostics). The reaction mixture comprised: master mix (LightCycler® 480 Probes Master; 6.4 mM MgCl₂) forward and reverse primer (0.5 μM final conc.), carboxyfluorescein (FAM) labelled probe (0.2 μM final conc.), template DNA (5 μl) and nuclease free dH₂O to a final volume of 20 μl. The cycling parameters consisted of 10 minutes incubation at 95° C. to activate the polymerase (Taq), 50 cycles of 95° C. for 10 seconds and 60° C. for 30 s, followed by a cooling step at 40° C. for 10 s. The temperature transition rate (ramp rate on the LightCycler® 480 Instrument) was 4.4° C./s while heating and 2.2° C./s while cooling.

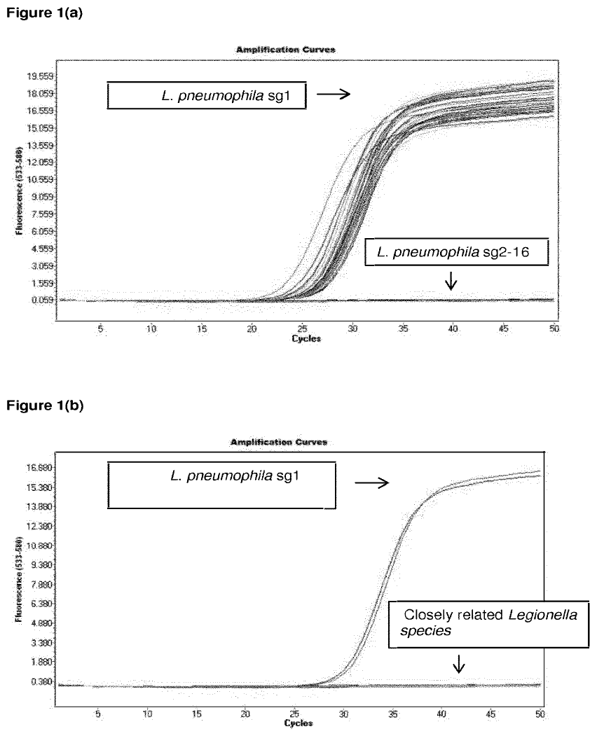

As demonstrated (FIG. 1(a)), the assay targeting a region of the lpg0768 gene detected all 12 Legionella pneumophila serogroup 1 strains tested for (Table 1 (a)); but the assay did not detect Legionella pneumophila serogroup 2-16 strains, or the no template control. The remaining Legionella species tested for (Table 1 (b)) were not detected by the assay targeting a region of the lpg0768 gene (FIG. 1(b)).

### Example 2—Assay for the Detection of Legionella pneumophila

### In Silico Analysis

Publicly available smpB gene nucleotide sequence information was retrieved from the National Center for Biotechnology Information website (ncbi.nlm.nih.gov/) for Legionella pneumophila and aligned using ClustalW (ebi.ac.uk/Tools/msa/clustalw2/). Partial gene sequence was also generated for a number of culture collection strains using sequencing primers and then aligned. All publicly available and generated nucleotide sequences for Legionella pneumophila sequences were subsequently aligned which demonstrated sufficient nucleotide similarity to design oligonucleotide primers and probes.

In this example, the following exemplary real-time PCR assay primers and probe for the smpB gene nucleotide sequence (SEQ ID NO. 6) were used: smpB forward primer (SEQ ID NO. 7), smpB reverse primer (SEQ ID NO. 8), smpB probe (SEQ ID NO. 9). Optionally an alternative smpB reverse primer (SEQ ID No. 13) could be used. The smpB probe (SEQ ID NO. 9) comprised at least one marker, optionally at least one dye, optionally at least one fluorophore, optionally rhodamine (5′ ROX, Integrated DNA Technologies). Optionally, the smpB probe (SEQ ID NO. 9) comprised at least one fluorescence quenching dye, optionally a terminal quencher (3′ Iowa Black® RQ, Integrated DNA Technologies; or Black Hole Quencher 2®, LGC Biosearch).

### Nucleotide Sequencing

To generate a partial smpB gene nucleotide sequence for L. pneumophila, conventional PCR was performed using sequencing primers on a thermal cycler (iCycler iQ thermal cycler; Bio-Rad Laboratories Inc., California, USA). All reactions were carried out in a final volume of 50 μl, containing 5 μl 10× buffer (15 mM MgCl₂), 1 μl forward and reverse primers (0.2 μM final conc.), 2 μl DNA polymerase (Taq; 1 U/μl, Roche Diagnostics, Basel, Switzerland), 1 μl dNTP mix (10 mM deoxynucleoside triphosphate set; Roche Diagnostics), 5 μl of template DNA and 35 μl nuclease free water (Applied Biosystems/Ambion, Texas, USA). The cycling parameters consisted of initial denaturation at 95° C. for 5 minutes, followed by 35 cycles of denaturation at 95° C. (1 minutes), annealing at 50° C. (1 minutes), and extension at 72° C. (1 minutes), with a final elongation step at 72° C. for 10 minutes.

PCR products were visualised on a 1.5% agarose gel and subsequently purified using a PCR product purification kit (High Pure PCR Product Purification Kit, Roche Diagnostics). Purified PCR products were sent for sequencing externally (Sequiserve, Vaterstetten, Germany).

### DNA Isolation and Quantification

Genomic DNA from Legionella cultures were isolated using a modified procedure combining mechanical lysis (IDI lysis kit; Becton Dickenson, Canada) and purification using a DNA purification kit (quick gDNA kit, Zymo Research, Irvine, CA, USA). Briefly, a loop of culture was resuspended in 250 μl lysis buffer (IDI lysis buffer). The suspension was transferred to a cell lysis tube (GeneOhm) and bead beaten (Mini-Bead-Beater-16; Stratech, United Kingdom) for 3 minutes. After bead beating, 200 μl was transferred to a nucleic acid purification spin column (Zymo-Spin™ Column) in a collection tube and steps 2 to 5 of the procedure for purification of total DNA from cell suspensions were followed, according to the manufacturer's instructions.

### Bacterial Strains, Culture Media, and Growth Conditions

A panel of 26 Legionella pneumophila strains and 41 other Legionella species and 14 other bacteria were used in this study (Table 2 (a) and Table 2 (b)). These species and strains were purchased from the German Collection of Microorganisms and Cell Cultures (Deutsche Sammlung von Mikroorganismen und Zellkulturen GmbH, DSMZ. Braunschweig, Germany), the National Collection of Type Cultures (NCTC, Public Health England, Salisbury, United Kingdom), American Type Culture Collection (ATCC, provided by LGC standards, Middlesex United Kingdom) and Culture Collection, University of Göteborg, (CCUG, Sweden). All Legionella species and strains were cultured on BCYE media at 37° C. 18-24 hours or until sufficient growth was observed.

TABLE 2(a)
Legionella pneumophila strains used in this study

| Strain | Culture collection |
|---|---|
| Legionella pneumophila subsp. pneumophila-1 | DSM 7513 |
| Legionella pneumophila subsp. fraseri-4 | DSM 7514 |
| Legionella pneumophila subsp. pasculei-5 | DSM 7515 |
| Legionella pneumophila subsp. pneumophila-1 | NCTC 11191 |
| Legionella pneumophila subsp. pneumophila-2 | NCTC 11230 |
| Legionella pneumophila subsp. pneumophila-1 | NCTC 11231 |
| Legionella pneumophila subsp. pneumophila-3 | NCTC 11232 |
| Legionella pneumophila subsp. pneumophila-1 | NCTC 11286 |
| Legionella pneumophila subsp. pneumophila-6 | NCTC 11287 |
| Legionella pneumophila subsp. pneumophila-1 | NCTC 11378 |
| Legionella pneumophila subsp. pneumophila-1 | NCTC 11404 |
| Legionella pneumophila subsp. pneumophila-5 | NCTC 11417 |
| Legionella pneumophila subsp. pneumophila-7 | NCTC 11984 |
| Legionella pneumophila subsp. pneumophila-8 | NCTC 11985 |
| Legionella pneumophila subsp. pneumophila-9 | NCTC 11986 |
| Legionella pneumophila subsp. pneumophila-10 | NCTC 12000 |
| Legionella pneumophila subsp. pneumophila-14 | NCTC 12174 |
| Legionella pneumophila subsp. pneumophila-11 | NCTC 12179 |
| Legionella pneumophila subsp. pneumophila-12 | NCTC 12180 |
| Legionella pneumophila subsp. pneumophila-13 | NCTC 12181 |
| Legionella pneumophila subsp. pneumophila-1 | DSM 25019 |
| Legionella pneumophila subsp. pneumophila-1 | DSM 25071 |
| Legionella pneumophila subsp. pneumophila-1 | DSM 25199 |
| Legionella pneumophila subsp. pneumophila-1 | DSM 25200 |
| Legionella pneumophila subsp. pneumophila-1 | DSM 25213 |
| Legionella pneumophila subsp. pneumophila-1 | DSM 27564 |

*DSM = The German Collection of Microorganisms;
*NCTC = National Collection of Type Cultures

TABLE 2(b)
Other Legionella species tested in this study

| Species | culture collection number |
|---|---|
| Legionella anisa | DSM 17627 |
| Legionella birminghamensis | DSM 19232 |
| Legionella birminghamensis | ATCC 700709 |
| Legionella bozemanii | NCTC 11975 |
| Legionella bozemanii | DSM 16523 |
| Legionella cincinnatiensis | DSM 19233 |
| Legionella dumoffii | DSM 17625 |
| Legionella dumoffii | ATCC 33343 |
| Legionella erythra | DSM 17644 |
| Legionella feeleii-1 | DSM 17645 |
| Legionella feelii-2 | NCTC 11978 |
| Legionella gormanii | DSM 16641 |
| Legionella gormanii | ATCC 43769 |
| Legionella hackeliae-1 | DSM 19214 |
| Legionella hackeliae-2 | NCTC 11980 |
| Legionella jordanis | DSM 19212 |
| Legionella jordanis | ATCC 700762 |
| Legionella lansingensis | DSM 19556 |
| Legionella longbeachae-1 | DSM 10572 |
| Legionella maceachemii | DSM 16642 |
| Legionella miodadei | NCTC 11403 |
| Legionella oakridgensis | NCTC 11531 |
| Legionella oakridgensis | ATCC 700515 |
| Legionella parisiensis | DSM 19216 |
| Legionella wadworthii | NCTC 11532 |
| Legionella cherrii | DSM 19213 |
| Legionella jamestowniensis | DSM 19215 |
| Legionella londoniensis | DSM 21234 |
| Legionella taurinsis | CCUG 44901 |
| Legionella moravica | DSM 19234 |
| Legionella adelaidensis | DSM 19888 |
| Legionella donaldsonii | ATCC BAA-693 |
| Legionella gratiana | DSM 21233 |
| Legionella gresilensis | DSM 21218 |
| Legionella fairfieldensis | NCTC 12488 |
| Legionella israelensis | DSM 19235 |
| Legionella fallonii | CCUG 43887 |
| Legionella brunensis | DSM 19236 |
| Legionella busanensis | ATCC BAA-518 |
| Legionella quinlivanii | NCTC 12434 |
| Legionella rubrilicens | DSM 11884 |
| Burkholderia cepacia | DSM 7522 |
| Ralstonia pickettii | DSM 6297 |
| Serratia marcescens | DSM 30121 |
| Cupriavidus pauculus | DSM 17313 |
| Stenotrophomonas maltophilia | DSM 21874 |
| Pseudomonas maltophila | DSM 50071 |
| Klebsiella pneumoniae | DSM 12059 |
| Proteus mirabilis | DSM 4479 |
| Enterobacter aerogenes | DSM 2969 |
| Acinetobacter baumannii | LMG 984 |
| Staphylococcus aureus | DSM 11822 |
| Bacillus cereus | DSM 30584 |
| Clostridium difficile | DSM 1296 |
| Sphingomonas paucimobilis | DSM 1098 |

*DSM = The German Collection of Microorganisms;
*ATCC = American Type Culture Collection;
*NCTC = National Collection of Type Cultures;
*CCUG = Culture Collection, University of Göteborg

### Real-Time PCR

The Legionella pneumophila specific assay was then tested in a real-time PCR format against a panel of well characterised Legionella species to determine assay specificity (Inclusivity and Exclusivity testing) and suitability of the smpB gene nucleotide sequence as a diagnostics marker.

Real-time PCR was performed on a real-time PCR amplification device (LightCycler® 480 Instrument, Roche Diagnostics) using a real-time PCR probes kit (LightCycler® 480 Probes Master kit, Roche Diagnostics). The reaction mixture comprised: master mixture (LightCycler® 480 Probes Master) in 6.4 mM MgCl₂, forward and reverse primer (0.5 μM final conc.), carboxyfluorescein (FAM) labelled probe (0.2 μM final conc.), template DNA (5 μl) and nuclease free dH₂O to a final volume of 20 μl. The cycling parameters consisted of 10 minutes incubation at 95° C. to activate the polymerase (Taq), 50 cycles of 95° C. for 10 seconds and 60° C. for 30 s, followed by a cooling step at 40° C. for 10 s. The temperature transition rate, (ramp rate on the LightCycler® 480 Instrument) was 4.4° C./s while heating and 2.2° C./s while cooling.

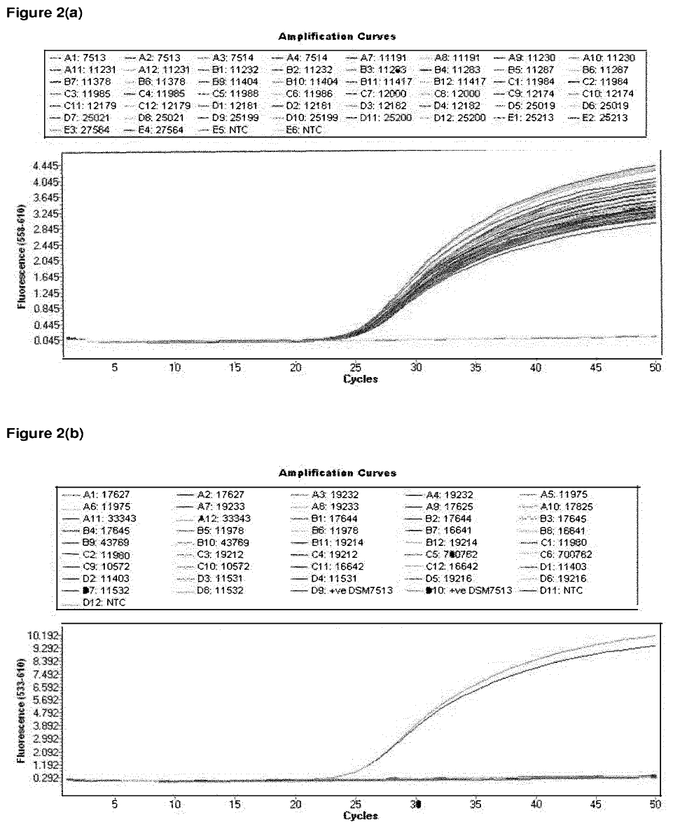

As demonstrated in FIG. 2(a), all 26 Legionella pneumophila strains tested for (Table 2 (a)) were detected by the L. pneumophila species assay targeting a region of the smpB gene and the no-template control was not detected by the assay. As shown in FIG. 2(b), the L. pneumophila species assay targeting a region of the smpB gene detected the one L. pneumophila isolate tested for and did not detect the 41 other Legionella species and other bacteria that were tested for (Table 2(b)).

### Example 3: An Internally Controlled Multiplex Real-Time PCR for the Detection and Identification of Legionella Species, Legionella pneumophila (Serogroup 1-16) and Legionella pneumophila Serogroup 1

### Diagnostic Target Identification

A number of housekeeping genes which are highly conserved throughout the Legionella genus were evaluated to identify, in silico, a potential diagnostic target which could be used to detect all members of this genus and for the detection of Legionella pneumophila strains. The optimal diagnostic target chosen in this study for detection of the Legionella genus was the ssrA gene. The ssrA gene codes for tmRNA and has been identified in all bacterial species. The function of tmRNA in bacteria is to rescue stalled ribosomes and to clear the cell of incomplete polypeptides. In silico alignment of publically available nucleotide sequences indicated that while sufficient nucleotide sequence heterogeneity exists between different Legionella species, sufficient nucleotide sequence similarity existed to design genus oligonucleotide primers and probes.

In this example, the following exemplary real-time PCR assay primers and probe for the ssrA gene nucleotide sequence (SEQ ID NO. 5) were used: ssrA forward primer (SEQ ID NO. 10), ssrA reverse primer (SEQ ID NO. 11), ssrA probe (SEQ ID NO. 12). The ssrA probe (SEQ ID NO. 12) comprised at least one marker, optionally at least one dye, optionally at least one fluorophore, optionally fluorescein (5′ 6-FAM, Integrated DNA Technologies). Optionally, the ssrA probe (SEQ ID NO. 12) comprised at least one fluorescence quenching dye, optionally an internal quencher (ZEN™ quencher, Integrated DNA Technologies), optionally a terminal quencher (3′ Iowa Black® FQ, Integrated DNA Technologies; or Black Hole Quencher 1°, LGC Biosearch).

By in silico alignment, it was demonstrated that there is perfect, or near-perfect, conservation of the each of the following nucleotide sequences: the exemplary ssrA forward primer (SEQ ID NO. 10), the exemplary ssrA reverse primer (SEQ ID NO. 11), and the exemplary ssrA probe (SEQ ID NO. 12); among the ssrA gene nucleotide sequences of the following Legionella species: L. pneumophilaATCC43290, L. pneumophila_ThunderBay, L. pneumophila_HL06041035, L. pneumophila_str.Philadelphia, L. pneumophila_Lens, L. pneumophila_Paris, L. pneumophila_Corby, L. pneumophila_2300/99Alcoy, L. pneumophila_Lorraine, L. longbeachaeD-4968, L. longbeachaeNSW150, L. fallonii_LLAP-10, L. hackeliae_LHA, L. oakridgensis_ATCC33761 L. micdadei_LMI, L. sainthelensi_ATCC35248, L. wadsworthiiDSM21896, L. cherrii_DSM19213, L. moravicaDSM19234, L. norrlandica, L. shakespeareiDSM23087, L. drancourtiiLLAP12 L. lansingensis_DSM19556, L. fairfieldensis_ATCC_49588, L. massiliensis_PRJEB110, and L. geestianaDSM21217.

The optimal diagnostic target chosen in this study for detection of the Legionella pneumophila was the smpB gene. The smpB gene encodes the RNA binding protein Small Protein B (SmpB) and has been identified in all bacterial species to date. It is considered an essential component of quality control in bacteria as it facilitates the binding of tmRNA to stalled ribosomes which in turn allows for the removal of incomplete polypeptides from the cell. In silico alignment of publically available nucleotide sequences indicated that sufficient nucleotide sequence similarity exists between different serogroups of Legionella pneumophila to allow for the design of species specific oligonucleotide primers and probes.

In this example, the following exemplary real-time PCR assay primers and probe for the smpB gene nucleotide sequence (SEQ ID NO. 6) were used: smpB forward primer (SEQ ID NO. 7), smpB reverse primer (SEQ ID NO. 8), smpB probe (SEQ ID NO. 9). Optionally an alternative smpB reverse primer (SEQ ID No. 13) could be used. The smpB probe (SEQ ID NO. 9) comprised at least one marker, optionally at least one dye, optionally at least one fluorophore, optionally rhodamine (5′ ROX, Integrated DNA Technologies). Optionally, the smpB probe (SEQ ID NO. 9) comprised at least one fluorescence quenching dye, optionally a terminal quencher (3′ Iowa Black® RQ, Integrated DNA Technologies; or Black Hole Quencher 2°, LGC Biosearch).

By in silico alignment, it was demonstrated that there is perfect, or near-perfect, conservation of the each of the following nucleotide sequences: the exemplary smpB forward sequencing primer (SEQ ID NO. 14), the exemplary smpB forward primer (SEQ ID NO. 7), the exemplary smpB reverse sequencing primer (SEQ ID NO. 15), the exemplary smpB reverse primer (SEQ ID NO. 8), and the exemplary smpB probe (SEQ ID NO. 9); among the smpB nucleotide sequences of the following Legionella species: L. pneumophila_Paris, L. pneumophila_Corby, L. pneumophila_Pneu, L. pneumophila_Lens, L. pneumophila_Alcoy, L. pneumophila_2868, L. pneumophila_3140, L. pneumophila_3194, L. pneumophila_00189, L. pneumophila_2829, L. pneumophila_NCTC11986, L. pneumophila_NCTC12000, L. pneumophila_NCTC12179, L. pneumophila_DSM7513, L. pneumophilaDSM7514, L. pneumophilaDSM7515, L. pneumophila_NCTC11191, L. pneumophila_NCTC11230, L. pneumophila_NCTC11232, L. pneumophila_NCTC11287, L. pneumophila_NCTC11984, L. pneumophila_NCTC11985.

Based on the evaluation of housekeeping genes performed in this study, it was not possible to identify a Legionella pneumophila serogroup 1 specific target. As such whole genome comparisons were performed to identify a novel diagnostics target in silico. Publicly available whole genome sequence information was retrieved from the National Center for Biotechnology Information website (ncbi.nlm.nih.gov/) for Legionella pneumophila serogroup 1 and Legionella pneumophila serogroup 6 and serogroup 12. Regions potentially unique to L. pneumophila serogroup 1 were identified using WEBACT (webact.org/). This resulted in a number of regions of difference in L. pneumophila serogroup 1. Each identified putative target nucleotide sequence was BLAST analysed (blast.ncbi.nlm.nih.gov/Blast.cgi) to determine if they were present in all L. pneumophila serogroup 1 and absent in all other L. pneumophila serogroups or closely related Legionella species. One region was present in all L. pneumophila serogroup 1 analysed with very low or no sequence similarity to other L. pneumophila serogroups or closely related Legionella species. This region was the lpg0768 gene (SEQ ID NOs 1 and 21-48) which encodes the spore coat polysaccharide biosynthesis protein E, NeuB. Closely related nucleotide sequences from Legionella pneumophila serogroup 1 strains were aligned using ClustalW (ebi.ac.uk/Tools/msa/clustalw2/).

By in silico alignment, it was demonstrated that there is partial, perfect, or near-perfect, conservation of the each of the following nucleotide sequences: the exemplary lpg0768forward sequencing primer (SEQ ID NO. 16), the exemplary lpg0768 forward primer (SEQ ID NO. 2), the exemplary lpg0768 reverse sequencing primer (SEQ ID NO. 17), the exemplary lpg0768 reverse primer (SEQ ID NO. 3), and the exemplary lpg0768 probe (SEQ ID NO. 4); among the lpg0768 nucleotide sequences of the following Legionella pneumophila serogroup 1 strains: gi|395129037 L. pneumophila_extended, L. pneumophila_str.Philadelphia1(sg1), L. pneumophila_str.Paris(sg1), L. pneumophila_LPE509 (SG1), gi|6688579 L. pneumophila(sg1), L. pneumophila_str.Lens(sg1), gi|410173912 L. pneumophila(sg1), L. pneumophila_str.Camperdown1(sg1), L. pneumophila_str.Heysham1(sg1), L. pneumophila_str.Uppsala3(sg1), L. pneumophila2300/99Alcoy(sg1), L. pneumophila_str.Corby(sg1), L. pneumophila_str.uppsala3.b(sg1), L. pneumophila_str.Lorraine(sg1), L. pneumophila_str.Goerlitz6543(sg1), L. pneumophila_str.HL06041035(sg1), L. pneumophila_str.Hextuple_3a(sg1), L. pneumophila_str.Hextuple_2q(sg1), L. pneumophila(sg1)_DSM7513, L. pneumophila(sg1)_NCTC11191, L. pneumophila(sg1)_NCTC11231, L. pneumophila(sg1)_NCTC11378, L. pneumophila(sg1)_NCTC11404, L. pneumophila(sg1)_DSM25019, L. pneumophila(sg1)_DSM25021, L. pneumophila(sg1)_DSM25199, L. pneumophila(sg1)_DSM25200, L. pneumophila(sg1)_DSM25213, and L. pneumophila(sg1)_DSM37564.

### Bacterial Strains, Culture Media, and Growth Conditions

A panel of 26 Legionella pneumophila strains and 41 other Legionella species were used in this study (Tables 2.1 and 2.2). These species and strains were purchased from the German Collection of Microorganisms and Cell Cultures (Deutsche Sammlung von Mikroorganismen und Zellkulturen GmbH, DSMZ. Braunschweig, Germany), the National Collection of Type Cultures (NCTC, Public Health England, Salisbury, United Kingdom), American Type Culture Collection (ATCC, provided by LGC standards, Middlesex United Kingdom) and Culture Collection, University of Göteborg, (CCUG, Sweden). All Legionella species and strains were cultured on BCYE media at 37° C. for 18-24 hours or until sufficient growth was observed.

### Genomic DNA Isolation and Quantification.

Genomic DNA from Legionella cultures were isolated using a modified procedure combining mechanical lysis (IDI lysis kit; Becton Dickenson, Canada) and purification using a DNA purification kit (quick gDNA kit; Zymo Research, Irvine, CA, USA). Briefly, a loop of culture was resuspended in 250 μl lysis buffer (IDI lysis buffer). The suspension was transferred to a cell lysis tube (GeneOhm) and bead beaten (Mini-Bead-Beater-16; Stratech, United Kingdom) for 3 minutes. After bead beating, 200 μl was transferred to a nucleic acid purification spin column (Zymo-Spin™ Column) in a collection tube and steps 2 to 5 of the procedure for purification of total DNA from cell suspensions were followed, according to the manufacturer's instructions.

### Nucleotide Sequencing

To generate partial smpB nucleotide sequence for L. pneumophila, conventional PCR was performed using the sequencing primers outlined in Table 3 (a) on a thermal cycler (iCycler iQ thermal cycler; Bio-Rad Laboratories Inc., California, USA). All reactions were carried out in a final volume of 50 μl, containing 5 μl 10× buffer (15 mM MgCl₂), 1 μl forward and reverse primers (0.2 μM final conc.), 2 μl DNA polymerase (Taq; 1 U/μl, Roche Diagnostics, Basel, Switzerland), 1 μl dNTP mix (10 mM deoxynucleoside triphosphate set; Roche Diagnostics), 5 μl of template DNA and 35 μl nuclease free water (Applied Biosystems/Ambion, Texas, USA). The cycling parameters consisted of initial denaturation at 95° C. for 5 minutes, followed by 35 cycles of denaturation at 95° C. (1 minutes), annealing at 50° C. (1 minutes), and extension at 72° C. (1 minutes), with a final elongation step at 72° C. for 10 minutes.

TABLE 3(a)
Oligonucleotides used in this study

| | | | | SEQ |
|---|---|---|---|---|
| | Target | | | ID |
| Name | gene | Function | 5′-3′ Sequence | NO. |
| LegGen-F1 | ssrA | Genus real- | GGCGACCTGGCTTC | 10 |
| | | time PCR | | |
| | | assay | | |
| | | forward | | |
| | | primer | | |
| LegGen-R1 | ssrA | Genus real- | TATGACCGTTGATTCGATACC | 11 |
| | | time PCR | | |
| | | assay | | |
| | | reverse | | |
| | | primer | | |
| LegGen-P1 | ssrA | Genus real- | /56-FAM/ACGTGGGTT/ZEN/ | 12 |
| | | time PCR | GCRAAACC/3IABkFQ/ | |
| | | assay probe | | |
| LgensmpB_F | smpB | L. | GATCAATACGAAGCAGGC | 14 |
| | | pneumophila | | |
| | | forward | | |
| | | Seqeuncing | | |
| | | primer | | |
| LgensmpB_R | smpB | L. | GCCATTCTCTGTCTTTGATC | 15 |
| | | pneumophila | | |
| | | reverse | | |
| | | Seqeuncing | | |
| | | primer | | |
| LegPneuF1 | smpB | L. | CACGTGATAATMAAATACGGT | 7 |
| | | pneumophila | | |
| | | real-time | | |
| | | PCR assay | | |
| | | forward | | |
| | | primer | | |
| LegPneuR1 | smpB | L. | TTCATCAATAGCTTGCGYG | 8 |
| | | pneumophila | | |
| | | real-time | | |
| | | PCR assay | | |
| | | reverse | | |
| | | primer | | |
| LegPneuR12 | smpB | L. | TTCATCAACAGCTTGCGTG | 13 |
| | | pneumophila | | |
| | | real-time | | |
| | | PCR assay | | |
| | | reverse | | |
| | | primer 2 | | |
| LegPneuP1 | smpB | L. | /56-ROXN/CYGCATCCACTCA | 9 |
| | | pneumophila | TTTTATTCCTGAT/3IAbRQSp/ | |
| | | real-time | | |
| | | PCR assay | | |
| | | probe | | |
| L.pneu(sg1)_Fseq | region 5′ | L. | CCAAGTAGGGTTTCTAACAATC | 16 |
| | lpg0768 | pneumophila | | |
| | | sg1 forward | | |
| | | Seqeuncing | | |
| | | primer | | |
| L.pneu(sg1)_Rseq | region 3′ | L. | GAGTCAGCCTTTTACAATTGG | 17 |
| | lpg0768 | pneumophila | | |
| | | sg1 reverse | | |
| | | Seqeuncing | | |
| | | primer | | |
| L.pneu(sg1)_F | lpg0768 | L. | AGCTCCCAATGCAATAGAC | 2 |
| | | pneumophila | | |
| | | sg1 real- | | |
| | | time PCR | | |
| | | assay | | |
| | | forward | | |
| | | primer | | |
| L. pneu(sg1)_R | lpg0768 | L. | GCAAAGCAGTTGAAACAGC | 3 |
| | | pneumophila | | |
| | | sg1 real- | | |
| | | time PCR | | |
| | | assay | | |
| | | reverse | | |
| | | primer | | |
| L. pneu(sg1)_P | lpg0768 | L. | /5HEX/CCAACTGGT/ZEN/ | 4 |
| | | pneumophila | AATGAAAAACATTGGT/3IABkFQ/ | |
| | | sg1 real- | | |
| | | time PCR | | |
| | | assay probe | | |
| SIACF1 | synthetic | IAC real- | ATGCCAGTCAGCATAAGGA | 18 |
| | construct | time PCR | | |
| | | assay | | |
| | | forward | | |
| | | primer | | |
| SIACF2 | synthetic | IAC real- | CAGACCTCTGGTAGGATGTAC | 19 |
| | construct | time PCR | | |
| | | assay | | |
| | | reverse | | |
| | | primer | | |
| SIACP1 | synthetic | IAC real- | 5Cy5/TCGGCACTA/TAO/ | 20 |
| | construct | time PCR | CCGACACGAAC/3IAbRQSp/ | |
| | | probe | | |

/56-FAM/ = 5′ 6-FAM (fluorescein) fluorophore;
/ZEN/ = ZEN ™ internal quencher, made by Integrated DNA Technologies;
/3IABkFQ/ = 3′ Iowa Black ® FQ terminal quencher, made by Integrated DNA Technologies;
/56-ROXN/ = 5′ ROX (carboxy-X-rhodamine) n-hydroxysuccinimide ester fluorophore;
/5HEX/ = 5′ HEX (hexachlorofluorescein) fluorophore;
/5Cy5/ = 5′ Cy5 (cyanine) fluorophore;
/TAO/ = TAO ™ internal quencher, made by Integrated DNA Technologies;
/3IAbRQSp/ = 3′ Iowa Black ® RQ terminal quencher, made by Integrated DNA Technologies.

To generate the lpg0768 gene nucleotide sequence for L. pneumophila serogroup 1 strains, conventional PCR was performed using the sequencing primers outlined in Table 3 (a) on the thermal cycler (iCycler iQ thermal cycler; Bio-Rad Laboratories Inc., California, USA). All reactions were carried out in a final volume of 50 μl, containing 5 μl 10× buffer (15 mM MgCl₂), 1 μl forward and reverse primers (0.2 μM final conc.), 2 μl DNA polymerase (Taq; 1 U/μl, Roche Diagnostics, Basel, Switzerland), 1 μl dNTP mix (10 mM deoxynucleoside triphosphate set; Roche Diagnostics), 5 μl of template DNA and 35 μl nuclease free water (Applied Biosystems/Ambion, Texas, USA). The cycling parameters consisted of initial denaturation at 95° C. for 5 minutes, followed by 35 cycles of denaturation at 95° C. (1 minutes), annealing at 50° C. (1 minutes), and extension at 72° C. (1 minutes), with a final elongation step at 72° C. for 10 minutes.

PCR products were visualised on a 1.5% agarose gel and subsequently purified using a PCR product purification kit (High Pure Product Purification Kit; Roche Diagnostics). Purified PCR products were sent for sequencing externally (Sequiserve, Vaterstetten, Germany).

### Assay Design

All oligonucleotide primers and probes used in this example were designed in accordance with published guidelines. All oligonucleotide primers and probes were purchased from Integrated DNA Technologies (Leuven, Belgium).

### Development of an Internal Amplification Control (IAC) for Real-Time PCR

An internal amplification control (IAC) was designed and incorporated in the multiplex assay designed to monitor for PCR inhibition, thermocycler malfunction, degradation of assay components etc. As this multiplex assay is designed for use on environmental samples, a synthetic construct of random sequence was identified to be the most suitable IAC. The synthetic construct sequence is not found in nature and as such the specific oligonucleotide primers and probes will not be mopped up by competing microorganisms commonly found in the environment.

In this example, the following exemplary real-time PCR assay primers and probe for the IAC were used: IAC synthetic construct forward primer (SEQ ID NO. 18), IAC synthetic construct reverse primer (SEQ ID NO. 19), IAC synthetic construct probe (SEQ ID NO. 20). The IAC synthetic construct probe (SEQ ID NO. 20) comprised at least one marker, optionally at least one dye, optionally at least one fluorophore, optionally cyanine (5′ Cy5™, Integrated DNA Technologies). Optionally, the synthetic construct probe (SEQ ID NO. 20) comprised at least one fluorescence quenching dye, optionally an internal quencher (TAO™ quencher, Integrated DNA Technologies), optionally a terminal quencher (3′ Iowa Black® RQ, Integrated DNA Technologies).

Titrations of synthetic DNA were performed to determine the optimum concentration of IAC target per reaction such that the IAC is always detected without impacting on detection of the primary Legionella targets in the multiplex. An internal control concentration of 1000 genome equivalents per reaction allowed for the detection of the IAC all real-time PCR experiments performed.

A multiplex PCR assay was produced using a combination of oligonucleotide primers and probes selected from: the oligonucleotide primers and probes listed in Table 3 (a), and the oligonucleotide primers and probes described in example 1, the oligonucleotide primers and probes described in example 2, and the oligonucleotide primers and probes for the IAC synthetic construct described in this example.

### Real-Time PCR

To determine the specificity of each of the diagnostics target used in this study, Monoplex real-time PCR was performed on a thermal cycler (LightCycler 480; Roche Diagnostics, Basel, Switzerland) using a probes kit (LightCycler® Probes Master kit; Roche Diagnostics). A final volume of 20 μl was used in each reaction, containing 2×PCR reaction master mixture (master mix) [LightCycler 480 Probes Master (6.4 mM MgCl₂)], forward and reverse primer (0.5 mM final conc.), FAM, HEX, ROX or CY5 labelled probe (0.2 mM final conc.), template DNA (2 μl) and the volume adjusted to 20 μl with the addition of nuclease free dH₂O. The cycling parameters consisted of incubation for 10 minutes at 95° C. to activate enzymes and DNA denaturation followed by 50 cycles of 95° C. for 10 seconds and 60° C. for 30 s, followed by a cooling step at 40° C. for 10 s. The temperature transition rate for all cycling steps was 4.4° C./s while heating and 2.2° C./s while cooling.

Multiplex real-time PCR reactions were carried out on a thermal cycler (LightCycler 480; Roche Diagnostics, Basel, Switzerland) using a probes kit (LightCycler® Probes Master kit; Roche Diagnostics). A final volume of 20 μl was used for each multiplex experiment. The optimised master mix contained 2×PCR reaction master mixture [LightCycler 480 Probes Master (6.4 mM MgCl₂)], forward and reverse primer (0.125-0.25 mM final conc.), FAM, HEX, ROX and CY5 labelled dyes (0.2-0.4 μM final conc.), template DNA (5 μl), IAC DNA 1 μl, and the volume adjusted to 20 μl with the addition of nuclease free dH₂O. The internal control DNA was diluted to contain 1000 genome equivalents per μl and all other DNAs were tested at a range of 10*3-10*4 genome equivalents per 5 μl. The cycling parameters used were the same as those used in a monoplex format outlined above. A colour compensation file was generated using the technical note outlined in the Advanced Software Functionalities of the operator manual.

### Sensitivity and Specificity of the Real-Time PCR Assays

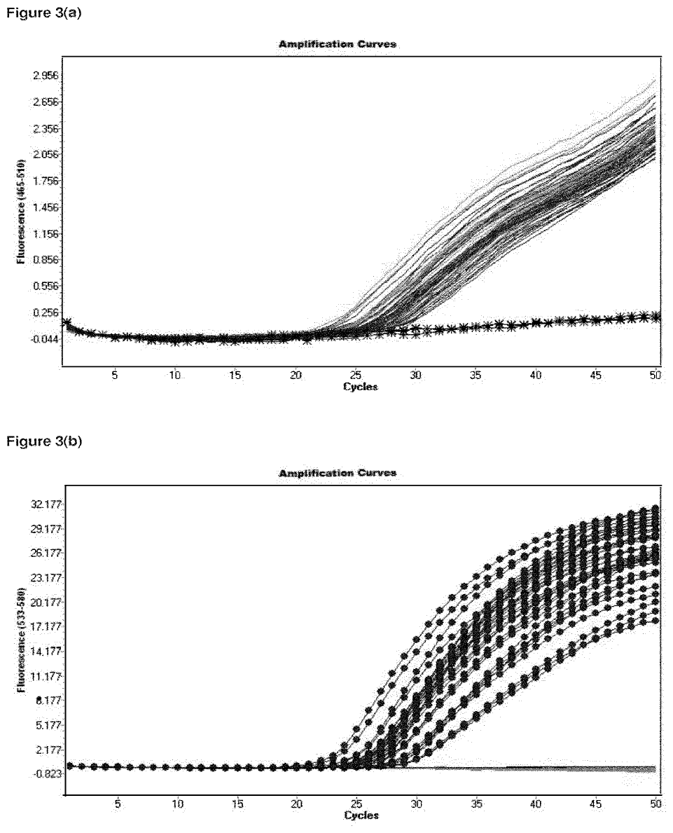

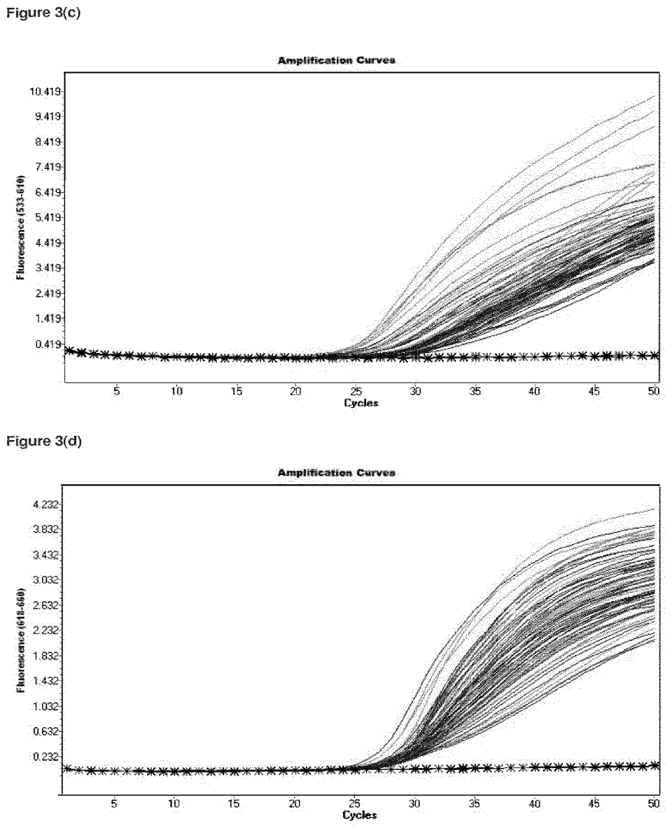

The specificity of each real-time PCR assay was confirmed both in monoplex and multiplex formats using the specificity panel listed in Table 3 (b) and Table 3 (c). Using the multiplex assay, all of the Legionella strains were detected in the Legionella genus assay targeting a region of the ssrA gene. A representation of this can be seen in FIG. 3(a), in which the negative control is represented by a line marked with stars. The L. pneumophila serogroup 1 specific assay targeting a region of the lpg0768 gene was specific for the detection of L. pneumophila serogroup 1 isolates. A representation of this can be seen in FIG. 3(b), in which the 12 L. pneumophila serogroup 1 strains tested are represented by lines marked with circles. All 12 L. pneumophila serogroup 1 strains were detected by the assay. The L. pneumophila species assay targeting a region of the smpB gene was specific for the detection of the 26 L. pneumophila strains tested for and the 41 other Legionella species were not detected by this assay. A representation of this can be seen in FIG. 3(c), in which the negative control is represented by a line marked with stars. The specificity of the IAC assay was tested using the full specificity panel (Table 3 (b) and Table 3 (c)) and was specific for the synthetic construct. As the IAC assay is designed using a non-competitive approach, when spiked into the master mix a positive signal should always be observed in the Cy5 channel. A representation of this can be seen in FIG. 3(d), in which the no template control is represented by a line marked with stars. The analytical specificity of this multiplex real-time PCR assay is 100%.

To determine the sensitivity of the multiplex real-time PCR assay developed, serial dilutions ranging from 10*4-1 cell equivalent of L. pneumophila were tested in triplicate. The limit of detection of the multiplex assay is between 1˜10 genome equivalents.

TABLE 3(b)
Legionella pneumophila strains used in this study

| Strain | Culture collection | Number | Serogroup |
|---|---|---|---|
| Legionella pneumophila | DSM | 7513 | 1 |
| Legionella pneumophila | NCTC | 11191 | 1 |
| Legionella pneumophila | NCTC | 11231 | 1 |
| Legionella pneumophila | NCTC | 11286 | 1 |
| Legionella pneumophila | NCTC | 11378 | 1 |
| Legionella pneumophila | NCTC | 11404 | 1 |
| Legionella pneumophila | DSM | 25019 | 1 |
| Legionella pneumophila | DSM | 25021 | 1 |
| Legionella pneumophila | DSM | 25199 | 1 |
| Legionella pneumophila | DSM | 25200 | 1 |
| Legionella pneumophila | DSM | 25213 | 1 |
| Legionella pneumophila | DSM | 27564 | 1 |
| Legionella pneumophila | NCTC | 11230 | 2 |
| Legionella pneumophila | NCTC | 11232 | 3 |
| Legionella pneumophila | DSM | 7414 | 4 |
| Legionella pneumophila | DSM | 7415 | 5 |
| Legionella pneumophila | NCTC | 11417 | 5 |
| Legionella pneumophila | NCTC | 11287 | 6 |
| Legionella pneumophila | NCTC | 11984 | 7 |
| Legionella pneumophila | NCTC | 11985 | 8 |
| Legionella pneumophila | NCTC | 11986 | 9 |
| Legionella pneumophila | NCTC | 12000 | 10 |
| Legionella pneumophila | NCTC | 12179 | 11 |
| Legionella pneumophila | NCTC | 12180 | 12 |
| Legionella pneumophila | NCTC | 12181 | 13 |
| Legionella pneumophila | NCTC | 12174 | 14 |

*DSM = The German Collection of Microorganisms;
*NCTC = National Collection of Type Cultures

TABLE 3(c)
Other Legionella species tested in this study

| Strain | Source |
|---|---|
| Legionella anisa | DSM 17627 |
| Legionella birminghamensis | DSM 19232 |
| Legionella birminghamensis | ATCC 700709 |
| Fluoribacter bozemanae (Legionella bozemanii) | DSM 16523 |
| Fluoribacter bozemanae-2 | NCTC 11975 |
| Legionella cincinnatiensis | DSM 19233 |
| Fluoribacter dumoffii (Legionella dumoffii) | DSM 17625 |
| Fluoribacter dumoffii | ATCC 33343 |
| Legionella erythra | DSM 17644 |
| Legionella feeleii-1 | DSM 17645 |
| Legionella feeleii-2 | NCTC 11978 |
| Flouribacter gormanii (Legionella gormanii) | DSM 16641 |
| Fluoribacter gormanii | ATCC 43769 |
| Legionella hackeliae-1 | DSM 19214 |
| Legionella hackeliae-2 | NCTC 11980 |
| Legionella jordanis | DSM 19212 |
| Legionella jordanis | ATCC 700762 |
| Legionella lansingensis | DSM 19556 |
| Legionella longbeachae-1 | DSM 10572 |
| Legionella maceachwerii | DSM 10572 |
| Tatlockia micdadei (Legionella micdadei) | NCTC 11403 |
| Legionella oakridgensis | NCTC 11531 |
| Legionella oakridgensis | ATCC 700515 |
| Legionella parisiensis | DSM 19216 |
| Legionella wadsworthii | NCTC 11532 |
| Legionella adelaidensis | DSM 19888 |
| Legionella brunensis | DSM 19236 |
| Legionella busanesis | ATCC-BAA518 |
| Legionella cherrii | DSM 19213 |
| Legionella donaldsonii | ATCC BAA-693 |
| Legionella fairfieldensis | NCTC 12488 |
| Legionella fallonii | CCUG 43887 |
| Legionella gratiana | DSM 21233 |
| Legionella gresilensis | DSM 21218 |
| Legionella israelensi | DSM 19235 |
| Legionella jamestowniensis | DSM 19215 |
| Legionella londoniensis | DSM 21234 |
| Legionella moravica | DSM 19234 |
| Legionella quinlivanii-1 | NCTC 12433 |
| Legionella rubrilicens | DSM 11884 |
| Legionella taurinsis | CCUG 44901 |

*DSM = The German Collection of Microorganisms;
*ATCC = American Type Culture Collection;
*NCTC = National Collection of Type Cultures;
*CCUG = Culture Collection, University of Göteborg

### Example 5—Diagnostics Algorithm

For determining which Legionella species and/or strains are present in a sample, the user must take into account the combination of results observed for each channel of the real-time in vitro amplification instrument. This are set out in Table 4 below, and explained below.

TABLE 4
Result of multiplex PCRs associated with each diagnosis

| Test | Multiplex 1 | | | | |
|---|---|---|---|---|---|
| (Target) | (ssrA) | (lpg0768) | (smpB) | (IAC) | |
| | • | • | • | • | Legionella pneumophila |
| | | | | | serogroup 1 |
| | • | X | • | • | Legionella pneumophila |
| | | | | | serogroup 2-16 |
| | • | X | X | • | Legionella species other |
| | | | | | than Legionella |
| | | | | | pneumophila |
| | X | X | X | • | Legionella species not |
| | | | | | present |
| | X | X | X | X | Result invalid |

Key:
• = positive signal,
X = not detected

### Example 6: An Internally Controlled Multiplex Real-Time PCR for the Detection and Identification of Legionella Species, Legionella pneumophila (Serogroup 1-16) and Legionella pneumophila Serogroup 1

### Diagnostic Target Identification

A number of housekeeping genes which are highly conserved throughout the Legionella genus were evaluated to identify, in silico, a potential diagnostic target which could be used to detect all members of this genus and for the detection of Legionella pneumophila strains. The optimal diagnostic target chosen in this study for detection of the Legionella genus was the ssrA gene. The ssrA gene codes for tmRNA and has been identified in all bacterial species. The function of tmRNA in bacteria is to rescue stalled ribosomes and to clear the cell of incomplete polypeptides. In silico alignment of publically available nucleotide sequences indicated that while sufficient nucleotide sequence heterogeneity exists between different Legionella species, sufficient nucleotide sequence similarity existed to design genus oligonucleotide primers and probes.

In this example, the following exemplary real-time PCR assay primers and probe for the ssrA gene nucleotide sequence (SEQ ID NO. 5) were used: ssrA forward primer (SEQ ID NO. 10), ssrA reverse primer (SEQ ID NO. 11), ssrA probe (SEQ ID NO. 12). The ssrA probe (SEQ ID NO. 12) comprised at least one marker, optionally at least one dye, optionally at least one fluorophore, optionally fluorescein (5′ 6-FAM, Integrated DNA Technologies). Optionally, the ssrA probe (SEQ ID NO. 12) comprised at least one fluorescence quenching dye, optionally an internal quencher (ZEN™ quencher, Integrated DNA Technologies), optionally a terminal quencher (3′ Iowa Black® FQ, Integrated DNA Technologies; or Black Hole Quencher 1°, LGC Biosearch).

By in silico alignment, it was demonstrated that there is perfect, or near-perfect, conservation of the each of the following nucleotide sequences: the exemplary ssrA forward primer (SEQ ID NO. 10), the exemplary ssrA reverse primer (SEQ ID NO. 11), and the exemplary ssrA probe (SEQ ID NO. 12); among the ssrA gene nucleotide sequences of the following Legionella species: L. pneumophilaATCC43290, L. pneumophila_ThunderBay, L. pneumophila_HL06041035, L. pneumophila_str.Philadelphia, L. pneumophila_Lens, L. pneumophila_Paris, L. pneumophila_Corby, L. pneumophila_2300/99Alcoy, L. pneumophila_Lorraine, L. longbeachaeD-4968, L. longbeachaeNSW150, L. fallonii_LLAP-10, L. hackeliae_LHA, L. oakridgensis_ATCC33761 L. micdadei_LMI, L. sainthelensi_ATCC35248, L. wadsworthiiDSM21896, L. cherrii_DSM19213, L. moravicaDSM19234, L. norrlandica, L. shakespeareiDSM23087, L. drancourtiiLLAP12 L. lansingensis_DSM19556, L. fairfieldensis_ATCC_49588, L. massiliensis_PRJEB110, and L. geestianaDSM21217.

The optimal diagnostic target chosen in this study for detection of the Legionella pneumophila was the smpB gene. The smpB gene encodes the RNA binding protein Small Protein B (SmpB) and has been identified in all bacterial species to date. It is considered an essential component of quality control in bacteria as it facilitates the binding of tmRNA to stalled ribosomes which in turn allows for the removal of incomplete polypeptides from the cell. In silico alignment of publically available nucleotide sequences indicated that sufficient nucleotide sequence similarity exists between different serogroups of Legionella pneumophila to allow for the design of species specific oligonucleotide primers and probes.

In this example, the following exemplary real-time PCR assay primers and probe for the smpB gene nucleotide sequence (SEQ ID NO. 6) were used: smpB forward primer (SEQ ID NO. 49), smpB reverse primer (SEQ ID NO. 50), smpB probes (SEQ ID NO. 51 and SEQ ID NO. 52). The smpB probes (SEQ ID NO. 51 and SEQ ID NO. 52) comprised at least one marker, optionally at least one dye, optionally at least one fluorophore, optionally rhodamine (5′ ROX, Integrated DNA Technologies). Optionally, the smpB probes (SEQ ID NO. 51 and SEQ ID NO. 52) comprised at least one fluorescence quenching dye, optionally a terminal quencher (3′ Iowa Black® RQ, Integrated DNA Technologies; or Black Hole Quencher 2°, LGC Biosearch).

By in silico alignment, it was demonstrated that there is perfect, or near-perfect, conservation of the each of the following nucleotide sequences: the exemplary smpB forward sequencing primer (SEQ ID NO. 14), the exemplary smpB forward primer (SEQ ID NO. 49), the exemplary smpB reverse sequencing primer (SEQ ID NO. 15), the exemplary smpB reverse primer (SEQ ID NO. 50), and the exemplary smpB probes (SEQ ID NO. 51 and SEQ ID NO. 52); among the smpB nucleotide sequences of the following Legionella species: L. pneumophila_Paris, L. pneumophila_Corby, L. pneumophila_Pneu, L. pneumophila_Lens, L. pneumophila_Alcoy, L. pneumophila_2868, L. pneumophila_3140, L. pneumophila_3194, L. pneumophila_00189, L. pneumophila_2829, L. pneumophila_NCTC11986, L. pneumophila_NCTC12000, L. pneumophila_NCTC12179, L. pneumophila_DSM7513, L. pneumophilaDSM7514, L. pneumophilaDSM7515, L. pneumophila_NCTC11191, L. pneumophila_NCTC11230, L. pneumophila_NCTC11232, L. pneumophila_NCTC11287, L. pneumophila_NCTC11984, L. pneumophila_NCTC11985.

Based on the evaluation of housekeeping genes performed in this study, it was not possible to identify a Legionella pneumophila serogroup 1 specific target. As such whole genome comparisons were performed to identify a novel diagnostics target in silico. Publicly available whole genome sequence information was retrieved from the National Center for Biotechnology Information website (ncbi.nlm.nih.gov/) for Legionella pneumophila serogroup 1 and Legionella pneumophila serogroup 6 and serogroup 12. Regions potentially unique to L. pneumophila serogroup 1 were identified using WEBACT (webact.org/). This resulted in a number of regions of difference in L. pneumophila serogroup 1. Each identified putative target nucleotide sequence was BLAST analysed (blast.ncbi.nlm.nih.gov/Blast.cgi) to determine if they were present in all L. pneumophila serogroup 1 and absent in all other L. pneumophila serogroups or closely related Legionella species. One region was present in all L. pneumophila serogroup 1 analysed with very low or no sequence similarity to other L. pneumophila serogroups or closely related Legionella species. This region was the lpg0768 gene (SEQ ID NO. 1, 21-49) which encodes the spore coat polysaccharide biosynthesis protein E, NeuB. Closely related nucleotide sequences from Legionella pneumophila serogroup 1 strains were aligned using ClustalW (ebi.ac.uk/Tools/msa/clustalw2/).

By in silico alignment, it was demonstrated that there is partial, perfect, or near-perfect, conservation of the each of the following nucleotide sequences: the exemplary lpg0768forward sequencing primer (SEQ ID NO. 16), the exemplary lpg0768 forward primer (SEQ ID NO. 2), the exemplary lpg0768 reverse sequencing primer (SEQ ID NO. 17), the exemplary lpg0768 reverse primer (SEQ ID NO. 3), and the exemplary lpg0768 probe (SEQ ID NO. 4); among the lpg0768 nucleotide sequences of the following Legionella pneumophila serogroup 1 strains: gi|395129037 L. pneumophila_extended, L. pneumophila_str.Philadelphia1(sg1), L. pneumophila_str.Paris(sg1), L. pneumophila_LPE509 (SG1), gi|6688579 L. pneumophila(sg1), L. pneumophila_str.Lens(sg1), gi|410173912 L. pneumophila(sg1), L. pneumophila_str.Camperdown1(sg1), L. pneumophila_str.Heysham1(sg1), L. pneumophila_str.Uppsala3(sg1), L. pneumophila2300/99Alcoy(sg1), L. pneumophila_str.Corby(sg1), L. pneumophila_str.uppsala3.b(sg1), L. pneumophila_str.Lorraine(sg1), L. pneumophila_str.Goerlitz6543(sg1), L. pneumophila_str.HL06041035(sg1), L. pneumophila_str.Hextuple_3a(sg1), L. pneumophila_str.Hextuple_2q(sg1), L. pneumophila(sg1)_DSM7513, L. pneumophila(sg1)_NCTC11191, L. pneumophila(sg1)_NCTC11231, L. pneumophila(sg1)_NCTC11378, L. pneumophila(sg1)_NCTC11404, L. pneumophila(sg1)_DSM25019, L. pneumophila(sg1)_DSM25021, L. pneumophila(sg1)_DSM25199, L. pneumophila(sg1)_DSM25200, L. pneumophila(sg1)_DSM25213, and L. pneumophila(sg1)_DSM37564.

### Bacterial Strains, Culture Media, and Growth Conditions

A panel of 26 Legionella pneumophila strains and 41 other Legionella species were used in this study (Tables 2.1 and 2.2). These species and strains were purchased from the German Collection of Microorganisms and Cell Cultures (Deutsche Sammlung von Mikroorganismen und Zellkulturen GmbH, DSMZ. Braunschweig, Germany), the National Collection of Type Cultures (NCTC, Public Health England, Salisbury, United Kingdom), American Type Culture Collection (ATCC, provided by LGC standards, Middlesex United Kingdom) and Culture Collection, University of Göteborg, (CCUG, Sweden). All Legionella species and strains were cultured on BCYE media at 37° C. for 18-24 hours or until sufficient growth was observed.

### Genomic DNA Isolation and Quantification.

Genomic DNA from Legionella cultures were isolated using a modified procedure combining mechanical lysis (IDI lysis kit; Becton Dickenson, Canada) and purification using a DNA purification kit (quick gDNA kit; Zymo Research, Irvine, CA, USA). Briefly, a loop of culture was resuspended in 250 μl lysis buffer (IDI lysis buffer). The suspension was transferred to a cell lysis tube (GeneOhm) and bead beaten (Mini-Bead-Beater-16; Stratech, United Kingdom) for 3 minutes. After bead beating, 200 μl was transferred to a nucleic acid purification spin column (Zymo-Spin™ Column) in a collection tube and steps 2 to 5 of the procedure for purification of total DNA from cell suspensions were followed, according to the manufacturer's instructions.

### Nucleotide Sequencing

To generate partial smpB nucleotide sequence for L. pneumophila, conventional PCR was performed using the sequencing primers outlined in Table 5 (a) on a thermal cycler (iCycler iQ thermal cycler; Bio-Rad Laboratories Inc., California, USA). All reactions were carried out in a final volume of 50 μl, containing 5 μl 10× buffer (15 mM MgCl₂), 1 μl forward and reverse primers (0.2 μM final conc.), 2 μl DNA polymerase (Taq; 1 U/μl, Roche Diagnostics, Basel, Switzerland), 1 μl dNTP mix (10 mM deoxynucleoside triphosphate set; Roche Diagnostics), 5 μl of template DNA and 35 μl nuclease free water (Applied Biosystems/Ambion, Texas, USA). The cycling parameters consisted of initial denaturation at 95° C. for 5 minutes, followed by 35 cycles of denaturation at 95° C. (1 minutes), annealing at 50° C. (1 minutes), and extension at 72° C. (1 minutes), with a final elongation step at 72° C. for 10 minutes.

TABLE 5(a)
Oligonucleotides used in this study

| | | | | SEQ |
|---|---|---|---|---|
| | Target | | | ID |
| Name | gene | Function | 5′-3′ Sequence | NO. |
| LegGen-F1 | ssrA | Genus real- | GGCGACCTGGCTTC | 10 |
| | | time PCR | | |
| | | assay | | |
| | | forward | | |
| | | primer | | |
| LegGen-R1 | ssrA | Genus real- | TATGACCGTTGATTCGATACC | 11 |
| | | time PCR | | |
| | | assay | | |
| | | reverse | | |
| | | primer | | |
| LegGen-P1 | ssrA | Genus real- | /56-FAM/ACGTGGGTT/ZEN/ | 12 |
| | | time PCR | GCRAAACC/3IABkFQ/ | |
| | | assay probe | | |
| LgensmpB_F | smpB | L. | GATCAATACGAAGCAGGC | 14 |
| | | pneumophila | | |
| | | forward | | |
| | | Seqeuncing | | |
| | | primer | | |
| LgensmpB_R | smpB | L. | GCCATTCTCTGTCTTTGATC | 15 |
| | | pneumophila | | |
| | | reverse | | |
| | | Seqeuncing | | |
| | | primer | | |
| Legionella sg1-16 | smpB | L. | GATCAATACGAAGCAGGC | 49 |
| F2 | | pneumophila | | |
| | | real-time | | |
| | | PCR assay | | |
| | | forward | | |
| | | primer | | |
| Legionella sg1-16 | smpB | L. | CCTTGYCTTTCAACACTTCC | 50 |
| R2 | | pneumophila | | |
| | | real-time | | |
| | | PCR assay | | |
| | | reverse | | |
| | | primer | | |
| Legionella sg1-16 | smpB | L. | /56-ROXN/ | 51 |
| P2 | | pneumophila | CATCCACTCATTTTATTCCTGATCC | |
| | | real-time | /3IAbRQSp/ | |
| | | PCR assay | | |
| | | probe | | |
| Legionella sg1-16 | smpB | L. | /56-ROXN/ | 52 |
| P3 | | pneumophila | CATCTACTCATTTTATTCCTGATCC | |
| | | real-time | /3IAbRQSp/ | |
| | | PCR assay | | |
| | | probe | | |
| L. pneu(sg1)_Fseq | region 5′ | L. | CCAAGTAGGGTTTCTAACAATC | 16 |
| | lpg0768 | pneumophila | | |
| | | sg1 forward | | |
| | | Seqeuncing | | |
| | | primer | | |
| L. pneu(sg1)_Rseq | region 3′ | L. | GAGTCAGCCTTTTACAATTGG | 17 |
| | lpg0768 | pneumophila | | |
| | | sg1 reverse | | |
| | | Seqeuncing | | |
| | | primer | | |
| L.pneu(sg1)_F | lpg0768 | L. | AGCTCCCAATGCAATAGAC | 2 |
| | | pneumophila | | |
| | | sg1 real- | | |
| | | time PCR | | |
| | | assay | | |
| | | forward | | |
| | | primer | | |
| L. pneu(sg1)_R | lpg0768 | L. | GCAAAGCAGTTGAAACAGC | 3 |
| | | pneumophila | | |
| | | sg1 real- | | |
| | | time PCR | | |
| | | assay | | |
| | | reverse | | |
| | | primer | | |
| L. pneu(sg1)_P | lpg0768 | L. | /5HEX/CCAACTGGT/ZEN/ | 4 |
| | | pneumophila | AATGAAAAACATTGGT/3IABkFQ/ | |
| | | sg1 real- | | |
| | | time PCR | | |
| | | assay probe | | |
| SIACF1 | synthetic | IAC real- | ATGCCAGTCAGCATAAGGA | 18 |
| | construct | time PCR | | |
| | | assay | | |
| | | forward | | |
| | | primer | | |
| SIACF2 | synthetic | IAC real- | CAGACCTCTGGTAGGATGTAC | 19 |
| | construct | time PCR | | |
| | | assay | | |
| | | reverse | | |
| | | primer | | |
| SIACP1 | synthetic | IAC real- | 5Cy5/TCGGCACTA/TAO/ | 20 |
| | construct | time PCR | CCGACACGAAC/3IAbRQSp/ | |
| | | probe | | |

/56-FAM/ = 5′ 6-FAM (fluorescein) fluorophore;
/ZEN/ = ZEN ™ internal quencher, made by Integrated DNA Technologies;
/3IABkFQ/ = 3′ Iowa Black ® FQ terminal quencher, made by Integrated DNA Technologies;
/56-ROXN/ = 5′ ROX (carboxy-X-rhodamine) n-hydroxysuccinimide ester fluorophore;
/5HEX/ = 5′ HEX (hexachlorofluorescein) fluorophore;
/5Cy5/ = 5′ Cy5 (cyanine) fluorophore;
/TAO/ = TAO ™ internal quencher, made by Integrated DNA Technologies;
/3IAbRQSp/ = 3′ Iowa Black ® RQ terminal quencher, made by Integrated DNA Technologies.

To generate the lpg0768 gene nucleotide sequence for L. pneumophila serogroup 1 strains, conventional PCR was performed using the sequencing primers outlined in Table 5 (a) on the thermal cycler (iCycler iQ thermal cycler; Bio-Rad Laboratories Inc., California, USA). All reactions were carried out in a final volume of 50 μl, containing 5 μl 10× buffer (15 mM MgCl₂), 1 μl forward and reverse primers (0.2 μM final conc.), 2 μl DNA polymerase (Taq; 1 U/μl, Roche Diagnostics, Basel, Switzerland), 1 μl dNTP mix (10 mM deoxynucleoside triphosphate set; Roche Diagnostics), 5 μl of template DNA and 35 μl nuclease free water (Applied Biosystems/Ambion, Texas, USA). The cycling parameters consisted of initial denaturation at 95° C. for 5 minutes, followed by 35 cycles of denaturation at 95° C. (1 minutes), annealing at 50° C. (1 minutes), and extension at 72° C. (1 minutes), with a final elongation step at 72° C. for 10 minutes.

PCR products were visualised on a 1.5% agarose gel and subsequently purified using a PCR product purification kit (High Pure Product Purification Kit; Roche Diagnostics). Purified PCR products were sent for sequencing externally (Sequiserve, Vaterstetten, Germany).

### Assay Design

All oligonucleotide primers and probes used in this example were designed in accordance with published guidelines. All oligonucleotide primers and probes were purchased from Integrated DNA Technologies (Leuven, Belgium).

### Development of an Internal Amplification Control (IAC) for Real-Time PCR

An internal amplification control (IAC) was designed and incorporated in the multiplex assay designed to monitor for PCR inhibition, thermocycler malfunction, degradation of assay components etc. As this multiplex assay is designed for use on environmental samples, a synthetic construct of random sequence was identified to be the most suitable IAC. The synthetic construct sequence is not found in nature and as such the specific oligonucleotide primers and probes will not be mopped up by competing microorganisms commonly found in the environment.

In this example, the following exemplary real-time PCR assay primers and probe for the IAC were used: IAC synthetic construct forward primer (SEQ ID NO. 18), IAC synthetic construct reverse primer (SEQ ID NO. 19), IAC synthetic construct probe (SEQ ID NO. 20). The IAC synthetic construct probe (SEQ ID NO. 20) comprised at least one marker, optionally at least one dye, optionally at least one fluorophore, optionally cyanine (5′ Cy5™, Integrated DNA Technologies). Optionally, the synthetic construct probe (SEQ ID NO. 20) comprised at least one fluorescence quenching dye, optionally an internal quencher (TAO™ quencher, Integrated DNA Technologies), optionally a terminal quencher (3′ Iowa Black® RQ, Integrated DNA Technologies).

Titrations of synthetic DNA were performed to determine the optimum concentration of IAC target per reaction such that the IAC is always detected without impacting on detection of the primary Legionella targets in the multiplex. An internal control concentration of 1000 genome equivalents per reaction allowed for the detection of the IAC all real-time PCR experiments performed.

A multiplex PCR assay was produced using a combination of oligonucleotide primers and probes selected from: the oligonucleotide primers and probes listed in Table 5 (a), and the oligonucleotide primers and probes described in example 1, the oligonucleotide primers and probes described in example 2, and the oligonucleotide primers and probes for the IAC synthetic construct described in this example.

### Real-Time PCR

To determine the specificity of each of the diagnostics target used in this study, Monoplex real-time PCR was performed on a thermal cycler (LightCycler 480; Roche Diagnostics, Basel, Switzerland) using a probes kit (LightCycler® Probes Master kit; Roche Diagnostics). A final volume of 20 μl was used in each reaction, containing 2×PCR reaction master mixture (master mix) [LightCycler 480 Probes Master (6.4 mM MgCl₂)], forward and reverse primer (0.25 mM final conc. for Legionella genus and L. pneumophila sg1-16 assays; 0.50 mM final conc. for L. pneumophila sg1 assay; 0.3 mM final conc. for IAC assay), FAM (0.6 mM final conc.), HEX (0.2 mM final conc.), ROX (0.2 mM final conc.) or CY5 labelled probe (0.6 mM final conc.), template DNA (2 μl) and the volume adjusted to 20 μl with the addition of nuclease free dH₂O. The cycling parameters consisted of incubation for 10 minutes at 95° C. to activate enzymes and DNA denaturation followed by 50 cycles of 95° C. for 10 seconds and 60° C. for 30 s, followed by a cooling step at 40° C. for 10 s. The temperature transition rate for all cycling steps was 4.4° C./s while heating and 2.2° C./s while cooling.

Multiplex real-time PCR reactions were carried out on a thermal cycler (LightCycler 480; Roche Diagnostics, Basel, Switzerland) using a probes kit (LightCycler® Probes Master kit; Roche Diagnostics). A final volume of 20 μl was used for each multiplex experiment. The optimised master mix contained 2×PCR reaction master mixture [LightCycler 480 Probes Master (6.4 mM MgCl₂)], forward and reverse primer (0.25-0.5 mM final conc.), FAM, HEX, ROX and CY5 labelled dyes (0.2-0.6 μM final conc.), template DNA (5 μl), IAC DNA 1 μl, and the volume adjusted to 20 μl with the addition of nuclease free dH₂O. The internal control DNA was diluted to contain 1000 genome equivalents per μl and all other DNAs were tested at a range of 10*3-10*4 genome equivalents per 5 μl. The cycling parameters used were the same as those used in a monoplex format outlined above. A colour compensation file was generated using the technical note outlined in the Advanced Software Functionalities of the operator manual.

### Sensitivity and Specificity of the Real-Time PCR Assays

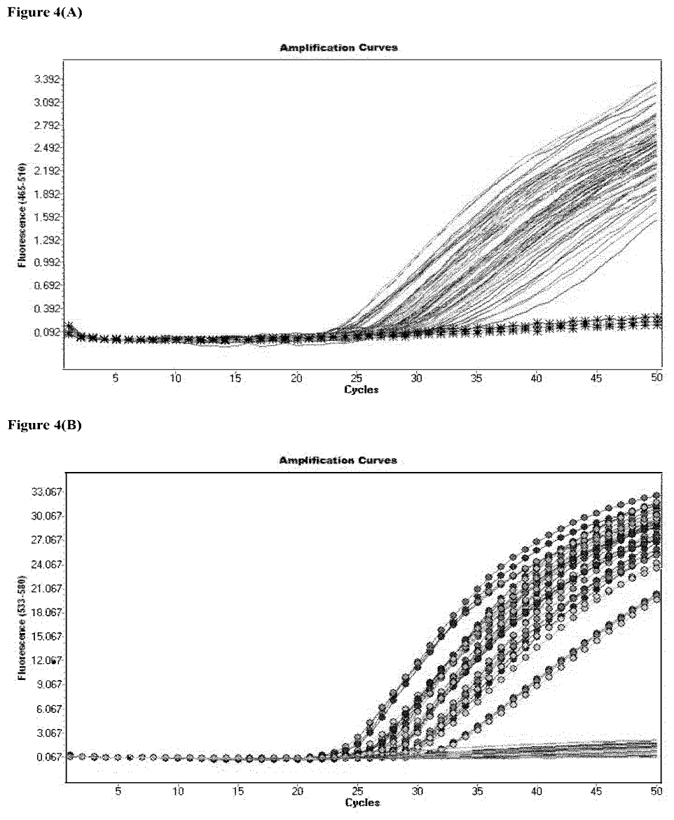

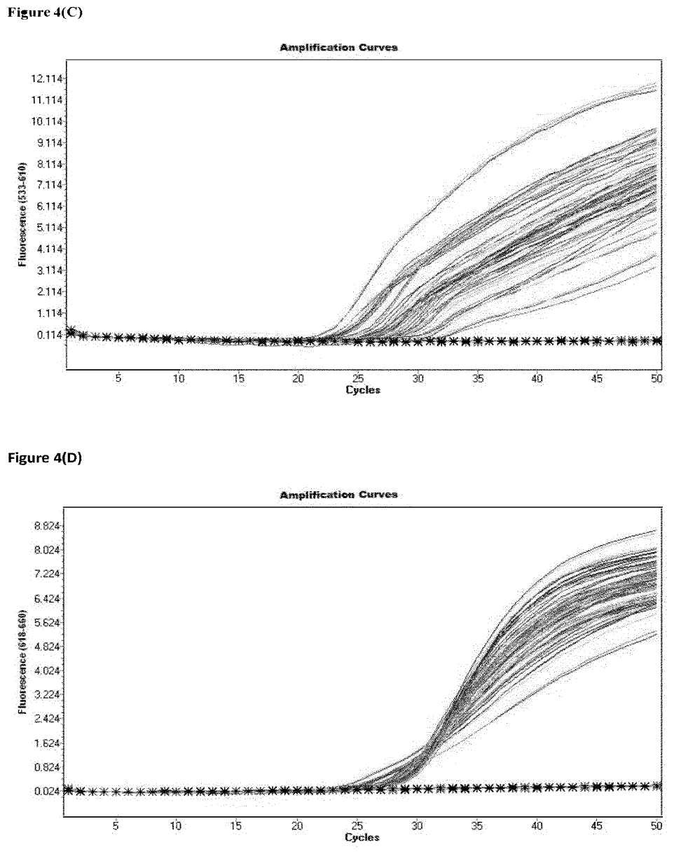

The specificity of each real-time PCR assay was confirmed both in monoplex and multiplex formats using the specificity panel listed in Table 5 (b) and Table 5 (c). Using the multiplex assay, all of the Legionella strains were detected in the Legionella genus assay targeting a region of the ssrA gene. A representation of this can be seen in FIG. 4(a), in which the negative control is represented by a line marked with stars. The L. pneumophila serogroup 1 specific assay targeting a region of the lpg0768 gene was specific for the detection of L. pneumophila serogroup 1 isolates. A representation of this can be seen in FIG. 4(b), in which the 12 L. pneumophila serogroup 1 strains tested are represented by lines marked with circles. All 12 L. pneumophila serogroup 1 strains were detected by the assay. The L. pneumophila species assay targeting a region of the smpB gene was specific for the detection of the 26 L. pneumophila strains tested for and the 41 other Legionella species were not detected by this assay. A representation of this can be seen in FIG. 4(c), in which the negative control is represented by a line marked with stars. The specificity of the IAC assay was tested using the full specificity panel (Table 5 (b) and Table 5 (c)) and was specific for the synthetic construct. As the IAC assay is designed using a non-competitive approach, when spiked into the master mix a positive signal should always be observed in the Cy5 channel. A representation of this can be seen in FIG. 4(d), in which the no template control is represented by a line marked with stars. The analytical specificity of this multiplex real-time PCR assay is 100%.

To determine the sensitivity of the multiplex real-time PCR assay developed, serial dilutions ranging from 10*4-1 cell equivalent of L. pneumophila were tested in triplicate. The limit of detection of the multiplex assay is between 1˜10 genome equivalents.

TABLE 5(b)
Legionella pneumophila strains used in this study

| Strain | Culture collection | Number | Serogroup |
|---|---|---|---|
| Legionella pneumophila | DSM | 7513 | 1 |
| Legionella pneumophila | NCTC | 11191 | 1 |
| Legionella pneumophila | NCTC | 11231 | 1 |
| Legionella pneumophila | NCTC | 11286 | 1 |
| Legionella pneumophila | NCTC | 11378 | 1 |
| Legionella pneumophila | NCTC | 11404 | 1 |
| Legionella pneumophila | DSM | 25019 | 1 |
| Legionella pneumophila | DSM | 25021 | 1 |
| Legionella pneumophila | DSM | 25199 | 1 |
| Legionella pneumophila | DSM | 25200 | 1 |
| Legionella pneumophila | DSM | 25213 | 1 |
| Legionella pneumophila | DSM | 27564 | 1 |
| Legionella pneumophila | NCTC | 11230 | 2 |
| Legionella pneumophila | NCTC | 11232 | 3 |
| Legionella pneumophila | DSM | 7414 | 4 |
| Legionella pneumophila | DSM | 7415 | 5 |
| Legionella pneumophila | NCTC | 11417 | 5 |
| Legionella pneumophila | NCTC | 11287 | 6 |
| Legionella pneumophila | NCTC | 11984 | 7 |
| Legionella pneumophila | NCTC | 11985 | 8 |
| Legionella pneumophila | NCTC | 11986 | 9 |
| Legionella pneumophila | NCTC | 12000 | 10 |
| Legionella pneumophila | NCTC | 12179 | 11 |
| Legionella pneumophila | NCTC | 12180 | 12 |
| Legionella pneumophila | NCTC | 12181 | 13 |
| Legionella pneumophila | NCTC | 12174 | 14 |

*DSM = The German Collection of Microorganisms;
*NCTC = National Collection of Type Cultures

TABLE 5(C)
Other Legionella species tested in this study

| Strain | Source |
|---|---|
| Legionella anisa | DSM 17627 |
| Legionella birminghamensis | DSM 19232 |
| Legionella birminghamensis | ATCC 700709 |
| Fluoribacter bozemanae (Legionella bozemanii) | DSM 16523 |
| Fluoribacter bozemanae-2 | NCTC 11975 |
| Legionella cincinnatiensis | DSM 19233 |
| Fluoribacter dumoffii (Legionella dumoffii) | DSM 17625 |
| Fluoribacter dumoffii | ATCC 33343 |
| Legionella erythra | DSM 17644 |
| Legionella feeleii-1 | DSM 17645 |
| Legionella feeleii-2 | NCTC 11978 |
| Flouribacter gormanii (Legionella gormanii) | DSM 16641 |
| Fluoribacter gormanii | ATCC 43769 |
| Legionella hackeliae-1 | DSM 19214 |
| Legionella hackeliae-2 | NCTC 11980 |
| Legionella jordanis | DSM 19212 |
| Legionella jordanis | ATCC 700762 |
| Legionella lansingensis | DSM 19556 |
| Legionella longbeachae-1 | DSM 10572 |
| Legionella maceachwerii | DSM 10572 |
| Tatlockia micdadei (Legionella micdadei) | NCTC 11403 |
| Legionella oakridgensis | NCTC 11531 |
| Legionella oakridgensis | ATCC 700515 |
| Legionella parisiensis | DSM 19216 |
| Legionella wadsworthii | NCTC 11532 |
| Legionella adelaidensis | DSM 19888 |
| Legionella brunensis | DSM 19236 |
| Legionella busanesis | ATCC-BAA518 |
| Legionella cherrii | DSM 19213 |
| Legionella donaldsonii | ATCC BAA-693 |
| Legionella fairfieldensis | NCTC 12488 |
| Legionella fallonii | CCUG 43887 |
| Legionella gratiana | DSM 21233 |
| Legionella gresilensis | DSM 21218 |
| Legionella israelensi | DSM 19235 |
| Legionella jamestowniensis | DSM 19215 |
| Legionella londoniensis | DSM 21234 |
| Legionella moravica | DSM 19234 |
| Legionella quinlivanii-1 | NCTC 12433 |
| Legionella rubrilicens | DSM 11884 |
| Legionella taurinsis | CCUG 44901 |

*DSM = The German Collection of Microorganisms;
*ATCC = American Type Culture Collection;
*NCTC = National Collection of Type Cultures;
*CCUG = Culture Collection, University of Göteborg

### Multiplex 1

Result Scenario for the Identification of L. pneumophila Serogroup 1

Using multiplex 1; if the ssrA genus assay, the lpg0768 assay, the smpB assay, and the IAC diagnostics assays each generate a positive signal, an organism selected from: L. pneumophila serogroup 1, is present in the sample.

Result Scenario for the Identification of L. pneumophila Serogroup 2-16

Using multiplex 1; if the ssrA genus assay, the smpB assay, and the IAC diagnostics assay each generate a positive signal, but the lpg0768 assay generates a negative signal, an organism selected from: L. pneumophila serogroup 2-16, is present in the sample.

Result Scenario for the Identification of Legionella Other than L. pneumophila

Using multiplex 1; if the ssrA genus assay and the IAC diagnostics assay generate a positive signal, but the smpB assay and the lpg0768 assay generate a negative signal, an organism selected from: Legionella species other than L. pneumophila, is present in the sample.

Result Scenario for when No Legionella are Present in a Sample

Using multiplex 1, if the ssrA genus assay, the lpg0768 assay, and the smpB assay are not detected, but the IAC diagnostics assays generates a positive signal, no organism of the Legionella genus is present in the sample

### Result Scenario for Invalid Result

Using multiplex 1, if no positive signal is observed for any diagnostics assay tested for, including the IAC diagnostics assay, the result is considered invalid and must be repeated.

### CONCLUSION

The multiplex real-time PCR assay described in this example is the first description of a real-time PCR diagnostics assay for the identification of the Legionella genus, L. pneumophila (2-16) and L. pneumophila serogroup 1 using the novel lpg0768 gene diagnostics targets. This multiplex real-time PCR diagnostics assay takes approximately one hour to perform after genomic DNA extraction and purification and has an analytical specificity of 100% and a sensitivity of less than 10 genome equivalents.

### REFERENCES

Halse, T. A., Edwards, J., Cunningham, P. L., Wolfgang, W. J., Dumas, N. B., Escuyer, V. E. & Musser, K. A. (2010) Combined Real-Time PCR and rpoB Gene Pyrosequencing for Rapid Identification of Mycobacterium tuberculosis and Determination of Rifampin Resistance Directly in Clinical Specimens. Journal of Clinical Microbiology, 48 (4), 1182-8.
Scheler, O., Glynn, B., Parkel, S., Palta, P., Toome, K., Kaplinski, L., Remm, M., Maher, M. & Kurg, A. (2009) Fluorescent labeling of NASBA amplified tmRNA molecules for microarray applications. BMC Biotechnology, 9 (45), Available from: bmcbiotechnol.biomedcentral.com/articles/10.1186/1472-6750-9-45 [Accessed: 23 Sep. 2016].

1.-33. (canceled)

34. A method for identifying the presence, absence, or quantity of Legionella pneumophila serogroup 1 in a sample, said method comprising:
contacting said sample with at least one primer or probe having a nucleic acid sequence defined by at least one of SEQ ID NO. 2, SEQ ID NO. 3, SEQ ID NO. 4, SEQ ID NO. 16, SEQ ID NO. 17 and the reverse complement each thereof, under conditions suitable for an in vitro amplification; and
detecting the presence, absence, or quantity of at least part of the nucleic acid sequence defined by SEQ ID NO. 1 or an expression product thereof;
wherein the presence, absence, or quantity of the at least part of the nucleic acid sequence defined by SEQ ID NO. 1 or expression product thereof correlates to the presence, absence, or quantity of Legionella pneumophila serogroup 1 in the sample.

35. The method of claim 34, wherein the method further comprises:
contacting the sample with at least one primer or probe having a nucleic acid sequence defined by at least one of SEQ ID NO. 7, SEQ ID NO. 8, SEQ ID NO. 9, SEQ ID NO. 13, SEQ ID NO. 14, SEQ ID NO. 15, SEQ ID NO. 49, SEQ ID NO. 50, SEQ ID NO 51, SEQ ID NO. 52, and the reverse complement each thereof, under conditions suitable for an in vitro amplification; and
detecting the presence, absence, or quantity of at least part of the nucleic acid sequence defined by SEQ ID NO. 6 or an expression product thereof;
wherein the presence, absence, or quantity of the at least part of the nucleic acid sequence defined by SEQ ID NO. 6 or expression product thereof correlates to the presence, absence, or quantity of Legionella pneumophila in the sample.

36. The method of claim 34, wherein the method further comprises:
contacting the sample with at least one primer or probe having a nucleic acid sequence defined by at least one of SEQ ID NO. 10, SEQ ID NO. 11, SEQ ID NO. 12 and the reverse complement each thereof, under conditions suitable for an in vitro amplification; and
detecting the presence, absence, or quantity of at least part of the nucleic acid sequence defined by SEQ ID NO. 5 or an expression product thereof;
wherein the presence, absence, or quantity of the at least part of the nucleic acid sequence defined by SEQ ID NO. 5 or expression product thereof correlates to the presence, absence, or quantity of Legionella gemus in the sample.

37. The method of claim 35, wherein the method further comprises:
contacting the sample with at least one primer or probe having a nucleic acid sequence defined by at least one of SEQ ID NO. 10, SEQ ID NO. 11, SEQ ID NO. 12 and the reverse complement each thereof, under conditions suitable for an in vitro amplification; and
detecting the presence, absence, or quantity of at least part of the nucleic acid sequence defined by SEQ ID NO. 5 or an expression product thereof;
wherein the presence, absence, or quantity of the at least part of the nucleic acid sequence defined by SEQ ID NO. 5 or expression product thereof correlates to the presence, absence, or quantity of Legionella gemus in the sample.

38. The method of claim 34, said method comprising:
contacting said sample with at least one primer having a nucleic acid sequence defined by at least one of SEQ ID NO. 2, SEQ ID NO. 3 and the reverse complement each thereof, under conditions suitable for an in vitro amplification;
contacting said sample with at least one probe having a nucleic acid sequence defined by SEQ ID NO. 4 and the reverse complement thereof, under conditions suitable for hybridization; and
detecting the presence, absence, or quantity of at least part of the nucleic acid sequence defined by SEQ ID NO. 1 or an expression product thereof;
wherein the presence, absence, or quantity of the at least part of the nucleic acid sequence defined by SEQ ID NO. 1 or expression product thereof correlates to the presence, absence, or quantity of Legionella pneumophila serogroup 1 in the sample.

39. The method of claim 34, said method comprising:
contacting said sample with at least one primer having a nucleic acid sequence defined by at least one of SEQ ID NO. 16, SEQ ID NO. 17 and the reverse complement each thereof, under conditions suitable for an in vitro amplification;
contacting said sample with at least one probe having a nucleic acid sequence defined by SEQ ID NO. 4 and the reverse complement thereof, under conditions suitable for hybridization; and
detecting the presence, absence, or quantity of at least part of the nucleic acid sequence defined by SEQ ID NO. 1 or an expression product thereof;
wherein the presence, absence, or quantity of the at least part of the nucleic acid sequence defined by SEQ ID NO. 1 or expression product thereof correlates to the presence, absence, or quantity of Legionella pneumophila serogroup 1 in the sample.

40. The method of claim 35, said method comprising:
contacting the sample with at least one primer or probe having a nucleic acid sequence defined by at least one of SEQ ID NO. 7, SEQ ID NO. 8 and the reverse complement each thereof, under conditions suitable for an in vitro amplification;
contacting said sample with at least one probe having a nucleic acid sequence defined by SEQ ID NO. 9 and the reverse complement thereof, under conditions suitable for hybridization; and
detecting the presence, absence, or quantity of at least part of the nucleic acid sequence defined by SEQ ID NO. 6 or an expression product thereof;
wherein the presence, absence, or quantity of the at least part of the nucleic acid sequence defined by SEQ ID NO. 6 or expression product thereof correlates to the presence, absence, or quantity of Legionella pneumophila in the sample.

41. The method of claim 35, said method comprising:
contacting the sample with at least one primer or probe having a nucleic acid sequence defined by at least one of SEQ ID NO. 7, SEQ ID NO. 13 and the reverse complement each thereof, under conditions suitable for an in vitro amplification;
contacting said sample with at least one probe having a nucleic acid sequence defined by SEQ ID NO. 9 and the reverse complement thereof, under conditions suitable for hybridization; and
detecting the presence, absence, or quantity of at least part of the nucleic acid sequence defined by SEQ ID NO. 6 or an expression product thereof;
wherein the presence, absence, or quantity of the at least part of the nucleic acid sequence defined by SEQ ID NO. 6 or expression product thereof correlates to the presence, absence, or quantity of Legionella pneumophila in the sample.

42. The method of claim 35, said method comprising:
contacting the sample with at least one primer or probe having a nucleic acid sequence defined by at least one of SEQ ID NO. 14, SEQ ID NO. 15 and the reverse complement each thereof, under conditions suitable for an in vitro amplification;
contacting said sample with at least one probe having a nucleic acid sequence defined by SEQ ID NO. 9 and the reverse complement thereof, under conditions suitable for hybridization; and
detecting the presence, absence, or quantity of at least part of the nucleic acid sequence defined by SEQ ID NO. 6 or an expression product thereof;
wherein the presence, absence, or quantity of the at least part of the nucleic acid sequence defined by SEQ ID NO. 6 or expression product thereof correlates to the presence, absence, or quantity of Legionella pneumophila in the sample.

43. The method of claim 35, said method comprising:
contacting the sample with at least one primer or probe having a nucleic acid sequence defined by at least one of SEQ ID NO. 49, SEQ ID NO. 50 and the reverse complement each thereof, under conditions suitable for an in vitro amplification;
contacting said sample with at least one probe having a nucleic acid sequence defined by at least one of SEQ ID NO. 51, SEQ ID NO. 52 and the reverse complement thereof, under conditions suitable for hybridization; and
detecting the presence, absence, or quantity of at least part of the nucleic acid sequence defined by SEQ ID NO. 6 or an expression product thereof;
wherein the presence, absence, or quantity of the at least part of the nucleic acid sequence defined by SEQ ID NO. 6 or expression product thereof correlates to the presence, absence, or quantity of Legionella pneumophila in the sample.

44. The method of claim 36, wherein the method further comprises:
contacting the sample with at least one primer or probe having a nucleic acid sequence defined by at least one of SEQ ID NO. 10, SEQ ID NO. 11 and the reverse complement each thereof, under conditions suitable for an in vitro amplification;
contacting said sample with at least one probe having a nucleic acid sequence defined by SEQ ID NO. 12 and the reverse complement thereof, under conditions suitable for hybridization; and
detecting the presence, absence, or quantity of at least part of the nucleic acid sequence defined by SEQ ID NO. 5 or an expression product thereof;
wherein the presence, absence, or quantity of the at least part of the nucleic acid sequence defined by SEQ ID NO. 5 or expression product thereof correlates to the presence, absence, or quantity of Legionella gemus in the sample.

45. The method of claim 37, wherein the method further comprises:
contacting the sample with at least one primer or probe having a nucleic acid sequence defined by at least one of SEQ ID NO. 10, SEQ ID NO. 11 and the reverse complement each thereof, under conditions suitable for an in vitro amplification;
contacting said sample with at least one probe having a nucleic acid sequence defined by SEQ ID NO. 12 and the reverse complement thereof, under conditions suitable for hybridization; and
detecting the presence, absence, or quantity of at least part of the nucleic acid sequence defined by SEQ ID NO. 5 or an expression product thereof;
wherein the presence, absence, or quantity of the at least part of the nucleic acid sequence defined by SEQ ID NO. 5 or expression product thereof correlates to the presence, absence, or quantity of Legionella gemus in the sample.

46. A Legionella detection kit comprising at least one primer or probe having a nucleic acid sequence defined by any of SEQ ID NOs. 2, 3, 4, 7, 8, 9, 10, 11, 12, 13, 14, 15, 16, 17, 49, 50, 51, and 52; and instructions for use.

47. A Legionella detection assay comprising a solid support and at least one primer or probe having the nucleic acid sequence defined by any of SEQ ID NOs. 2, 3, 4, 7, 8, 9, 10, 11, 12, 13, 14, 15, 16, 17, 49, 50, 51, and 52.
